(19) United States
(12) Patent Application Publication
(10) Pub. No.: US 2006/0293228 A1
(43) Pub. Date: Dec. 28, 2006

(54) Therapeutic compositions and methods using transforming growth factor-beta mimics

(76) Inventor: Rajendra S. Bhatnagar, Burlingame, CA (US)

Correspondence Address: WILSON SONSINI GOODRICH & ROSATI, 650 PAGE MILL ROAD, PALO ALTO, CA, 94304-1050, US

(21) Appl. No.: 11/166,260

(22) Filed: Jun. 24, 2005

(51) Int. Cl. A61K 38/18

(52) U.S. Cl. 514/12

(57) ABSTRACT

The invention provides methods, compositions, and kits for treating a variety of conditions using substances that display one or more activities of transforming growth factor-β (TGF-β mimics). Conditions include skin conditions, wounds, and the need for soft tissue augmentation. Compositions that may be used to treat such conditions contain a TGF-β mimic. Kits that may be used to treat such conditions contain a TGF-β mimic.

### BACKGROUND OF THE INVENTION

A variety of skin conditions exist for which no completely satisfactory treatment has been found. Examples of such conditions include psoriasis, dermatitis, acne, basal cell carcinoma, squamous cell carcinoma, and malignant melanoma. A similar situation exists for wound healing in that there is a need for methods of enhanced wound healing, especially in the case of chronic or extensive wounds. In addition, soft tissue augmentation is often necessary (e.g., sphincter augmentation) or desirable (e.g., for cosmetic purposes). Although progress has been made in the treatment of conditions of the skin, response to treatment is variable and often a condition is only marginally to moderately responsive to treatment. Treatments aimed at enhancing the healing of wounds, especially chronic wounds, while progressing, still do not lead to satisfactory outcomes; e.g., many diabetics still must suffer amputations as a result of diabetic ulcers that do not heal. Soft tissue augmentation is often associated with unwanted consequences such as hard encapsulation of an implant, or bursting or migration of implanted or injected augmentation materials. Hence, a need exists for improved therapeutic methods and compositions, in particular for treatment of skin conditions, enhanced wound healing, and soft tissue augmentation.

### SUMMARY OF THE INVENTION

The invention provides methods, compositions, and kits utilizing TGF-β mimics for therapeutic applications.

In one aspect the invention provides a method of therapy including administering to an individual in need of therapy an effective amount of a TGF-β mimic. In some embodiments the individual is suffering from a skin condition, a wound, or a need for soft tissue augmentation. In some embodiments, the TGF-β mimic has a structure with substantially the same atomic coordinates as those given in FIGS. 1A and 1B. In some embodiments, the TGF-β mimic is a peptide, for example, a peptide that comprises a sequence that is at least about 80% identical to a sequence selected from the group consisting of SEQ ID NOS: 1, 2, 3, 11, 13, and 14. In some embodiments the peptide comprises a sequence that is at least about 80% identical to SEQ ID NO: 1. In some embodiments, the TGF-β mimic is administered topically, for example, in a vehicle comprising a lotion. In some embodiments, the lotion comprises water, mineral oil, a polyethylene glycol, and lanolin alcohol. In some embodiments, the concentration of the TGF-β mimic in the lotion is about 0.0001% to about 0.01% by weight in the lotion. In some embodiments, the concentration of TGF-β mimic in the lotion is about 0.001% by weight. In some embodiments, the TGF-β mimic is administered in combination with one or more other active agents.

In some embodiments the individual suffers from a skin condition is selected from the group consisting of acne, dermatitis, psoriasis, actinic keratosis, urticaria, rosacea, mucositis, insect bites, hives, basal cell carcinoma, squamous cell carcinoma, papilloma, keratoacanthoma and malignant melanoma. In some embodiments, the skin condition is acne. In some embodiments, the skin condition is dermatitis.

In some embodiments, the individual suffers from the need for soft tissue augmentation, and wherein the TGF-β mimic is associated with a matrix. In some of these embodiments, the composition is administered in such a way as to result in an effect selected from the group consisting of cosmetic enhancement, reconstructive augmentation, sphincter enhancement, periodontal tissue repair, maxillofacial augmentation, nasal augmentation, and oral augmentation.

In some embodiments, the individual suffers from a wound. In some of these embodiments, the therapy results in wound healing with reduced scarring compared to without therapy, and in some embodiments, the therapy results in wound healing with no visible scarring.

In another aspect, the invention provides compositions.

In some embodiments the invention provides a composition for the treatment of a skin condition comprising a TGF-β mimic and another active ingredient effective in the treatment of said skin condition. In some embodiments, the composition is a composition for topical administration.

In some embodiments, the invention provides a TGF-β mimic in a vehicle at a concentration of greater than 0.00005% by weight. In some embodiments the invention provides a composition comprising a TGF-β mimic in a vehicle at a concentration of about 0.0001% to about 0.01% by weight. In some embodiments, the vehicle comprises water, mineral oil, a polyethylene glycol, and lanolin alcohol.

In yet another aspect, the invention provides kits. In some embodiments, the invention provides a kit for use in treatment of an individual comprising a composition comprising a TGF-β mimic and instructions for use of the TGF-β mimic in said treatment.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B depict the spatial coordinates for the atoms in a structure defining the biologically active TGFβ mimics useful in the methods of the invention.

### DETAILED DESCRIPTION OF THE INVENTION

The present invention provides methods, compositions, and kits for the treatment of various conditions with transforming growth factor-beta (TGF-β) mimics. In one aspect, the invention provides methods utilizing TGF-β mimics. In some embodiments, the invention provides methods for the treatment of skin conditions, including skin aging, acne, dermatitis, psoriasis, urticaria, carcinoma, and malignant melanoma, utilizing TGF-β mimics. In some embodiments, the invention provides methods for enhancing wound healing. In some embodiments, skin conditions or wounds are treated by topical application of a TGF-β mimic. In another aspect, the invention provides methods of treatment of conditions requiring soft tissue augmentation by use of TGF-β mimics, often in combination with a matrix, including augmentation of sphincters and cosmetic augmentation. In yet another aspect, the invention provides compositions suitable for treatment of skin conditions, for enhancing wound healing, or for soft tissue augmentation. In some embodiments the compositions are suitable for topical application. In still yet another aspect, the invention provides kits for the treatment of skin conditions, treatment of conditions requiring soft tissue augmentation.

I. TGF-β Mimics

The methods and compositions of the invention relate to TGF-β mimics. A “TGF-β mimic,” as that term is used herein, includes synthetic or naturally-occurring compounds. It can be any suitable molecule, such as a peptide, peptidomimetic, peptide nucleic acid, antibody, or small or large inorganic or organic molecule, or a mixture of two or more of these types of compounds (e.g., compound that contains peptide and peptidomimetic portions), that is capable of assuming a conformation that causes it to have at least one activity of TGF-β, as measured by one or more of the assays described herein. This conformation may be a conformation that is substantially similar to or the same as the structure of a region of TGF-β, as discussed more fully below. Alternatively, this conformation may be any suitable conformation that results in an effect similar to a natural TGF-β, even if the conformation is not similar to that of a natural TGF-β.

A. Structure and Activity

Examples of TGF-β mimics are described in, e.g., U.S. Pat. Nos. 5,661,127; 5,780,436; and 6,638,912. These include compounds that are structurally or biologically analogous to a region of TGF-β and mimic the conformation recognized by TGF-β binding species (e.g., TGF-β receptors).

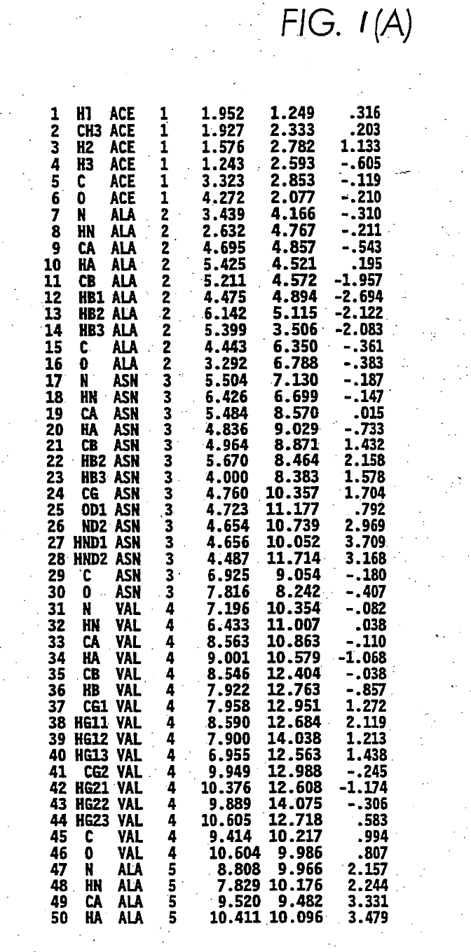

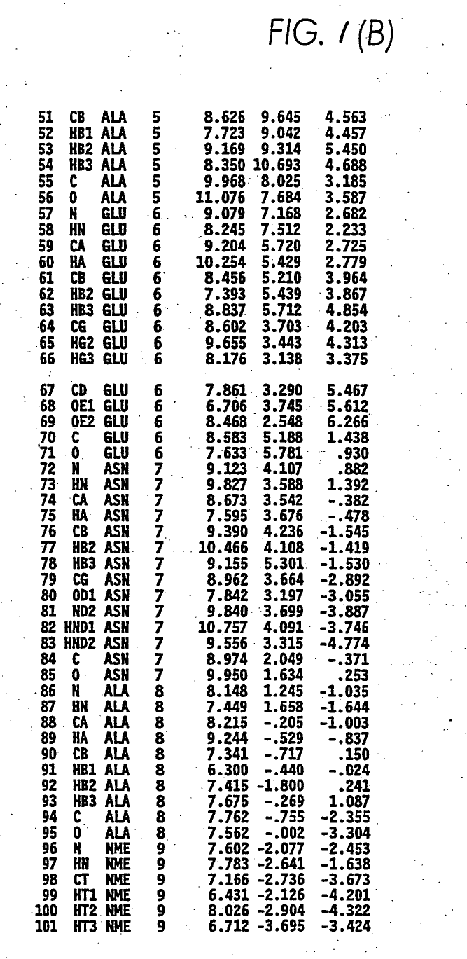

FIGS. 1A and 1B set out the coordinates for the atoms in a structure defining the biologically active TGF-β mimics useful in the methods of the invention. The coordinate computations were carried out using as a model compound, cytomodulin, described in U.S. Pat. No. 5,661,127, having the amino acid sequence Ala-Asn-Val-Ala-Glu-Asn-Ala (SEQ ID NO: 1); these coordinates were obtained by use of the AMBER and MIDAS programs. Structures with substantially these same coordinates (by “substantially” is meant to include up to about a 15% variance) will generate the desired surfaces and generally display the biological activities useful in the methods of the invention; presence of biological activity can be confirmed by the assays discussed below. The allowance for an amount of some variance is due to mobility and adaptability of fit between ligand and TGF-β receptor. Thus, such structures are considered to be TGF-β mimics for purposes of this invention.

Assays that may be used to assess TGF-β activity include those that measure the ability to promote anchorage independent growth of normal fibroblasts, for example, the growth and colony formation by NRK49 F fibroblasts in soft agar. Other assays that may be used to determine TGF-β activity include the inhibition of DNA synthesis in Mv-1-Lu mink lung epithelial cells, the induction of increased expression of type I collagen in primary cultures of neonatal human dermal fibroblasts, and/or induction of TGF-β synthesis.

Several amino acid sequences have been found to be TGF-β mimics, and are exemplary of TGF-β mimics useful in the methods provided herein. A TGF-β mimic thus may include an amino acid sequence as defined below.

The term “amino acid” as used herein means an organic compound containing both a basic amino group and an acidic carboxyl group. Included within this term are natural amino acids (e.g., generally the L-amino acids), and amino acids and imino acids which are known to occur biologically in free or combined form but usually do not occur in proteins. Included within this term also are modified and unusual amino acids, such as those disclosed in, for example, Roberts and Vellaccio (1983) The Peptides, 5: 342-429 (e.g., D-amino acids). In addition, the term “amino acid” also includes other non-naturally occurring amino acids besides the D-amino acids, which are functional equivalents of the naturally-occurring amino acids. Such non-naturally-occurring (also referred to herein as “unnatural amino acids”) amino acids include, for example, norleucine (“Nle”), norvaline (“Nva”), β-Alanine, L- or D-naphthalanine, ornithine (“Orn”), homoarginine (homoArg) and others well known in the peptide art, such as those described in M. Bodanzsky, Principles of Peptide Synthesis, 1st and 2nd revised ed., Springer-Verlag, New York, N.Y., 1984 and 1993, and Stewart and Young, Solid Phase Peptide Synthesis, 2nd ed., Pierce Chemical Co., Rockford, Ill., 1984. Amino acids and amino acid analogs can be purchased commercially (Sigma Chemical Co.; Advanced Chemtech; RSP; Bachem; or ChemImpex) or synthesized using methods known in the art.

“Natural amino acids” include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Natural non-protein amino acids include, but are not limited to arginosuccinic acid, citrulline, cysteine sulfinic acid, 3,4-dihydroxyphenylalanine, homocysteine, homoserine, ornithine, 3-monoiodotyrosine, 3,5-diiodotryosine, 3,5,5′-triiodothyronine, and 3,3′,5,5′-tetraiodothyronine. Modified or unusual amino acids which can be used to practice the invention include, but are not limited to, D-amino acids, hydroxylysine, 4-hydroxyproline, an N-CBZ-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N,N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

Standard three- and one-letter abbreviations for natural amino acid residues or amino acids apply throughout the specification unless otherwise indicated.

Unnatural amino acids that fall within the scope of this invention are by way of example and without limitation:

2-aminobutanoicacid, 2-aminopentanoic acid, 2-aminohexanoic acid, 2-aminoheptanoicacid, 2-aminooctanoic acid, 2-aminononanoic acid, 2-aminodecanoic acid, 2-aminoundecanoic acid, 2-amino-3,3-dimethylbutanoic acid, 2-amino-4,4-dimethylpentanoic acid, 2-amino-3-methylhexanoic acid, 2-amino-3-methylheptanoic acid, 2-amino-3-methyloctanoic acid, 2-amino-3-methylnonanoic acid, 2-amino-4-methylhexanoic acid, 2-amino-3-ethylpentanoic acid, 2-amino-3,4-dimethylpentanoic acid, 2-amino-3,5-dimethylhexanoic acid, 2-amino-3,3-dimethylpentanoic acid, 2-amino-3-ethyl-3-methylpentanoic acid, 2-amino-3,3-diethylpentanoic acid, 2-amino-5-methylhexanoic acid, 2-amino-6-methylheptanoic, 2-amino-7-methyloctanoic, 2-amino-2-cyclopentylacetic, 2-amino-2-cylcohexylacetic acid, 2-amino-2-(1-methylcylcohexyl)acetic acid, 2-amino-2-(2-methyl-1-methylcylcohexyl)acetic acid, 2-amino-2-(3-methyl-1-methylcylcohexyl)acetic acid, 2-amino-2-(4-methyl-methylcylcohexyl)acetic acid, 2-amino-2-(1-ethylcycolhexyl)acetic acid, 2-amino-3-(cyclohexyl)propanoic acid, 2-amino-4-(cyclohexyl)butanoic acid, 2-amino-3-(1-adamantyl)propanoic acid, 2-amino-3-butenoic acid, 2-amino-3-methyl-3-butenoic acid, 2-amino-4-pentenoic acid, 2-amino-4-hexenoic acid, 2-amino-5-heptenoic acid, 2-amino-4-methyl-4-hexenoic acid, 2-amino-5-methyl-4-hexenoic acid, 2-amino-4-methy-5-hexenoic acid, 2-amino-6-heptenoic acid, 2-amino-3,3,4-trimethyl-4-pentenoic acid, 2-amino-4-chloro-4-pentenoic, 2-amino-4,4-dichloro-3-butenoic acid, 2-amino-3-(2-methylenecyclopropyl)-propanoic acid, 2-amino-2-(2-cyclopentenyl)acetic acid, 2-amino-2-(cyclohexenyl)acetic acid, 2-amino-3-(2-cyclopentenyl)propanoic acid, 2-amino-3-(3-cyclopentenyl)propanoic acid, 2-amino-3-(1-cyclohexyl)propanoic acid, 2-amino-2-(1-cyclopentenyl)acetic acid, 2-amino-2-(1-cylcohexyl)acetic acid, 2-amino-2-(1-cylcoheptenyl)acetic acid, 2-amino-2-(1-cyclooctenyl)acetic acid, 2-amino-3-(1-cycloheptenyl)propanoic acid, 2-amino-3-(1,4-cyclohexadienyl)propanoic acid, 2-amino-3-(2,5-cyclohexadienyl)propanoic acid, 2-amino-2-(7-cycloheptatrienyl)acetic acid, 2-amino-4,5-hexadienoic acid, 2-amino-3-butynoic acid, 2-amino-4-pentyoic acid, 2-amino-4-hexynoic acid, 2-amino-4-hepten-6-ynoic acid, 2-amino-3-fluoropropanoic acid, 2-amino-3,3,3-trifluoropropanoic acid, 2-amino-3-fluorobutanoic acid, 2-amino-3-fluoropentanoic acid, 2-amino-3-fluorohexanoic acid, 2-amino-3,3-difluorobutanoic acid, 2-amino-3,3-difluoro-3-phenylpropanoic acid, 2-amino-3-perfluoroethylpropanoic acid, 2-amino-3-perfluoropropylpropanoic acid, 2-amino-3-fluoro-3-methylbutanoic acid, 2-amino-5,5,5-trifluoropentanoic acid, 2-amino-3-methyl-4,4,4-trifluorobutanoic acid, 2-amino-3-trifluoromethyl4,4,4-trifluorobutanoic acid, 2-amino-3,3,4,4,5,5-heptafluoropentanoic acid, 2-amino-3-methyl-5-fluoropentanoic acid, 2-amino-3-methyl-4-fluoropentanoic acid, 2-amino-5,5-difluorohexanoic acid, 2-amino-4-(fluoromethyl)-5-fluoropentanoic acid, 2-amino-4-trifluoromethyl-5,5,5-trifluoropentanoic acid, 2-amino-3-fluoro-3-methylbutanoic acid, 2-amino-3-fluoro-3-phenylpentanoic acid, 2-amino-2-(1-fluorocyclopentyl)acetic acid, 2-amino-2-(1-fluorocyclohexyl)acetic acid, 2-amino-3-chloropropanoic acid acid, 2-amino-3-chlorobutanoic acid acid, 2-amino-4,4-dichlorobutanoic acid acid, 2-amino-4,4,4-trichlorobutanoic acid, 2-amino-3,4,4-trichlorobutanoic acid, 2-amino-6-chlorohexanoic acid, 2-amino-4-bromobutanoic acid, 2-amino-3-bromobutanoic acid, 2-amino-3-mercaptobutanoic acid, 2-amino-4-mercaptobutanoic acid, 2-amino-3-mercapto-3,3-dimethylpropanoic acid, 2-amino-3-mercapto-3-methylpentanoic acid, 2-amino-3-mercaptopentanoic acid, 2-amino-3-mercapto-4-methylpentanoic acid, 2-amino-3-methyl-4-mercaptopentanoic acid, 2-amino-5-mercapto-5-methylhexanoic acid, 2-amino-2-(1-mercaptocyclobutyl)acetic acid, 2-amino-2-(1-mercaptocyclopentyl)acetic acid, 2-amino-2-(1-mercaptocyclohexyl)acetic acid, 2-amino-5-(methylthio)pentanoic acid, 2-amino-6-(methylthio)hexanoic acid, 2-amino-4-methylthio-3-phenylbutanoic acid, 2-amino-5-ethylthio-5-methylpentanoic acid, 2-amino-5-ethylthio-3,5,5-trimethylpentanoic acid, 2-amino-5-ethylthio-5-phenylpentanoic acid, 2-amino-5-ethylthio-5-pentanoic acid, 2-amino-5-butylthio-5-methylpentanoic acid, 2-amino-5-butylthio-3,5,5-trimethylpentanoic acid, 2-amino-5-butylthio-5-phenylpentanoic acid, 2-amino-5-(butylthio)pentanoic acid, 2-amino-3-methy4-hydroselenopentanoic acid, 2-amino-4-methylselenobutanoic acid, 2-amino-4-ethylselenobutanoic acid, 2-amino-4-benzylselenobutanoic acid, 2-amino-3-methyl-4-(methylseleno)butanoic acid, 2-amino-3-(aminomethylseleno)propanoic acid, 2-amino-3-(3-aminopropylseleno)propanoic acid, 2-amino-4-methyltellurobutanoic acid, 2-amino-4-hydroxybutanoic acid, 2-amino-4-hydroxyhexanoic acid, 2-amino-3-hydroxypentanoic acid, 2-amino-3-hydroxyhexanoic acid, 2-amino-3methyl-4-hydroxybutanoic acid, 2-amino-3-hydroxy-3-methylbutanoic acid, 2-amino-6-hydroxyhexanoic acid, 2-amino-4-hydroxyhexanoic acid, 2-amino-3-hydroxy-4-methylpentanoic acid, 2-amino-3-hydroxy-3-methylpentanoic acid, 2-amino-4-hydroxy-3,3-dimethylbutanoic acid, 2-amino-3-hydroxy4-methylpentanoic acid, 2-amino-3-hydroybutanedioic acid, 2-amino-3-hydroxy-3-phenyl-propanoic acid, 2-amino-3-hydroxy-3-(4-nitrophenyl)propanoic acid, 2-amino-3-hydroxy-3-(3-pyridyl)propanoic acid, 2-amino-2-(1-hydroxycyclopropyl) acetic acid, 2-amino-3-(1-hydroxycyclohexyl)propanoic acid, 2-amino-3-hydroxy-3-phenylpropanoic acid, 2-amino-3-hydroxy-3-[3-bis (2-chloroethyl)aminophenyl]propanoic acid, 2-amino-3-hydroxy-3-(3,4-dihydroxyphenyl)propanoic acid, 2-amino-3-hydroxy-3-(3,4-methylenedioxyphenyl)propanoic acid, 2-amino-4-fluoro-3-hydroxybutanoic acid, 2-amino-4,4,4-trichloro-3-hydroxybutanoic acid, 2-amino-3-hydroxy-4-hexynoic acid, 2-amino-3,4-dihydroxybutanoic acid, 2-amino-3,4,5,6-tetrahydroxyhexanoic acid, 2-amino-4,5-dihydroxy-3-methylpentanoic acid, 2-amino-5,6-dihydroxyhexanoic acid, 2-amino-5-hydroxy-4-(hydroxymethyl)pentanoic acid, 2-amino-4,5-dihydroxy4-(hydroxymethyl)pentanoic acid, 2-amino-3-hydroxy-5-benzyloxypentanoic acid, 2-amino-3-(2-aminoethoxy)propanoic acid, 2-amino-4-(2-aminoethoxy)butanoic acid, 2-amino-4-oxobutanoic acid, 2-amino-3-oxobutanoic acid, 2-amino-4-methyl-3-oxopentanoic acid, 2-amino-3-phenyl-3-oxopropanoic acid, 2-amino-4-phenyl-3-oxobutanoic acid, 2-amino-3-methyl-4-oxopentanoic acid, 2-amino-4-oxo-4-(4-hydroxyphenyl)butanoic acid, 2-amino-4-oxo-4-(2-furyl)butanoic acid, 2-amino-4-oxo-4-(2-nitrophenyl)butanoic acid, 2-amino-4-oxo-4-(2-amino-4-chlorophenyl)butanoic acid, 2-amino-3-(4-oxo-1-cyclohexenyl)propanoic acid, 2-amino-3-(4-oxocyclohexanyl)propanoic acid, 2-amino-3-(2,5-dimethyl-3,6-dioxo-1,4-cyclohexadienyl)propanoic acid, 2-amino-3-( 1-hydroxy-5-methyl-7-oxo-cyclohepta-1,3,5-trien-2-yl)propanoic acid, 2-amino-3-(1-hydroxy-7-oxo-cyclohepta-1,3,5-trien-3-yl)propanoic acid, 2-amino-3-(1-hydroxy-7-oxo-cyclohepta-1,3,5-trien-4-yl)propanoic acid, 2-amino-4-methoxy-3-butenoic acid, 2-amino-4-(2-aminoethoxy)-3-butenoic acid, 2-amino-4-(2-amino-3-hydroxypropyl)-3-butenoic acid, 2-amino-2-(4-methoxy-1,4-cyclohexadienyl)acetic acid, 2-amino-3,3-diethoxypropanoic acid, 2-amino-4,4-dimethylbutanoic acid, 2-amino-2-(2,3-epoxycyclohexyl)acetic acid, 2-amino-3-(2,3-epoxycyclohexy)propanoic acid, 2-amino-8-oxo-9,10-epoxydecanoic acid, 2-amino-propanedioic acid, 2-amino-3-methylbutanedioic acid, 2-amino-3,3-dimethylbutanedioic acid, 2-amino-4-methylpentanedioic acid, 2-amino-3-methylpentanedioic acid, 2-amino-3-phenylpentanedioic acid, 2-amino-3-hydroxypentanedioic acid, 2-amino-3-carboxypentanedioic acid, 2-amino-4-ethylpentanedioic acid, 2-amino-4-propylpentanedioic acid, 2-amino-4-isoamylpentanedioic acid, 2-amino-4-phenylpentanedioic acid, 2-amino-hexanedioic acid, 2-amino-heptanedioic acid, 2-amino-decanedioic acid, 2-amino-octanedioic acid, 2-amino-dodecanedioic acid, 2-amino-3-methylenebutanedioic acid, 2-amino-4-methylenepentanedioic acid, 2-amino-3-fluorobutanedioic acid, 2-amino-4-fluoropentanedioic acid, 2-amino-3,3-difluorobutanedioic acid, 2-amino-3-chloropentanedioic acid, 2-amino-3-hydroxybutanedioic acid, 2-amino-4-hydroxypentanedioic acid, 2-amino-4-hydroxyhexanedioic acid, 2-amino-3,4-dihydroxypentanedioic acid, 2-amino-3-(3-hydroxypropyl)butanedioic acid, 2-amino-3-(1-carboxy-4-hydroxy-2-cyclodienyl)propanoic acid, 2-amino-3-(aceto)butanedioic acid, 2-amino-3-cyanobutanedioic acid, 2-amino-3-(2-carboxy-6-oxo-6H-pyranyl)propanoic acid, 2-amino-3-carboxybutanedioic acid, 2-amino-4-carboxypentanedioic acid, 3-amido-2-amino-3-hydroxypropanoic acid, 3-amido-2-amino-3-methylpropanoic acid, 3-amido-2-amino-3-phenylpropanoic acid, 3-amido-2,3-diaminopropanoic acid, 3-amido-2-amino-3-[N-(4-hydroxyphenyl)amino]propanoic acid, 2,3-diaminopropanoic acid, 2,3-diaminobutanoic acid, 2,4-diaminobutanoic acid, 2,4-diamino-3-methylbutanoic acid, 2,4-diamino-3-phenylbutanoic acid, 2-amino-3-(methylamino)butanoic acid, 2,5-diamino-3-methylpentanoic acid, 2,7-diaminoheptanoic acid, 2,4-diaminoheptanoic acid, 2-amino-2-(2-piperidyl)acetic acid, 2-amino-2-(1-aminocyclohexyl)acetic acid, 2,3-diamino-3-phenylpropanoic acid, 2,3-diamino-3-(4-hydroxyphenyl)propanoic acid, 2,3-diamino-3-(4-methoxyphenyl)propanoic acid, 2,3-diamino-3-[4-(N,N′-dimethyamino)phenyl]propanoic acid, 2,3-diamino-3-(3,4-dimethoxyphenyl)propanoic acid, 2,3-diamino-3-(3,4-methylenedioxyphenyl)propanoic acid, 2,3-diamino-3-(4-hydroxy-3-methoxyphenyl)propanoic acid, 2,3-diamino-3-(2-phenylethyl)propanoic acid, 2,3-diamino-3-propylpropanoic acid, 2,6-diamino-4-hexenoic acid, 2,5-diamino-4-fluoropentanoic acid, 2,6-diamino-5-fluorohexanoic acid, 2,6-diamino-4-hexynoic acid, 2,6-diamino-5,5-difluorohexanoic acid, 2,6-diamino-5,5-dimethylhexanoic acid, 2,5-diamino-3-hydroxypentanoic acid, 2,6-diamino-3-hydroxyhexanoic acid, 2,5-diamino-4-hydroxypentanoic acid, 2,6-diamino-4-hydroxyhexanoic acid, 2,6-diamino-4-oxohexanoic acid, 2,7-diaminooctanedioic acid, 2,6-diamino-3-carboxyhexanoic acid, 2,5-diamino-4-carboxypentanoic acid, 2-amino-4-(2-(N,N′-diethylamino)ethyl)pentandioic acid, 2-amino-4-(N,N′-diethylamino)pentandioic acid, 2-amino-4-(N-morpholino)pentandioic acid, 2-amino-4-(N,N′-bis(2-chloroethyl)amino)pentandioic acid, 2-amino-4-(N,N′-bis(2-hydroxyethyl)amino)pentandioic acid, 2,3,5-triaminopentanoic acid, 2-amino-3-(N-(2-aminethyl)amino)propanoic acid, 2-amino-3-((2-aminoethyl)seleno)propanoic acid, 2-amino-3-[(2-aminoethyl)thio]propanoic acid, 2-amino-4-aminooxybutanoic acid, 2-amino-5-hydroxyaminopentanoic acid, 2-amino-5-[N-(5-nitro-2-pyrimidinyl)amino]pentanoic acid, 2-amino-4-[(7-nitro-2,1,3-benzoxadiazol-4-yl)amino]butanoic acid, 2-amino-3-guanidinopropanoic acid, 2-amino-3-guanidinobutanoic acid, 2-amino-4-guanidobutanoic acid, 2-amino-6-guanidohexanoic acid, 2-amino-6-ureidohexanoic acid, 2-amino-3-(2-iminoimidiazolin-4-yl)propanoic acid, 2-amino-2-(2-iminohexahydropyrinidin-4-yl)acetic acid, 2-amino-3-(2-iminohexahydropyrimidiny-4-yl)propanoic acid, 2-amino-4-fluoro-5-guanidopentanoic acid, 2-amino-4-hydroxy-5-guanidopentanoic acid, 2-amino-4-guanidooxybutanoic acid, 2-amino-6-aminohexanoic acid, 2-amino-5-(N-acetimidoylamino)pentanoic acid, 1-aminocyclopropanecarboxylic acid, 1-amino-4-ethylcyclpropanecarboxylic acid, 1-aminocyclopentanecarboxylic acid, 1-aminocyclopentanecarboxylic acid, 1-amino-2,2,5,5-tetramethyl-cyclohexanecarboxylic acid, 1-aminocydoheptanecarboxylic acid, 1-aminocyclononanecarboxylic acid, 2-aminoindan-2-carboxylic acid, 2-aminonorbornane-2-carboxylic acid, 2-amino-3-phenylnorbornane-2-carboxylic acid, 3-aminotetrahydrothiophene-3-carboxylic acid, 1-amino-1,3-cyclohexanedicarboxylic acid, 3-aminopyrrolidine-3-carboxylic acid, 1,4-diaminocyclohexanecarboxylic acid, 6-alkoxy-3-amino-1,2,3,4-tetrahydrocarbazole-3-carboxylic acid, 2-aminobenzobicyclo[2,2,2]octane-2-carboxylic acid, 2-arinoindan-2-carboxylic acid, 1-amino-2-(3,4-dhydroxyphenyl)cyclopropanecarboxylic acid, 5,6-dialkoxy-2-aminoindane-2-carboxylic acid, 4,5-dihydroxy-2-aminoindan-2-caroxylic acid, 5,6-dihydroxy-2-aminotetralin-2-carboxylic acid, 2-amino-2-cyanoacetic acid, 2-amino-3-cyanopropanoic acid, 2-amino-4-cyanobutanoic acid, 2-amino-5-nitropentanoic acid, 2-amino-6-nitrohexanoic acid, 2-amino-4-aminooxybutanoic acid, 2-amino-3-(N-nitrosohydroxyamino)propanoic acid, 2-amino-3-ureidopropanoic acid, 2-amino-4-ureidobutanoic acid, 2-amino-3-phosphopropanoic acid, 2-amino-3-thiophosphopropanoic acid, 2-amino-4-methanephosphonylbutanoic acid, 2-amino-3-(trimethylsilyl)propanoic acid, 2-amino-3-(dimethyl(trimethylsilylmethylsilyl)propanoic acid, 2-amino-2-phenylacetic acid, 2-amino-2-(3-chlorophenyl)acetic acid, 2-amino-2-(4-chlorophenyl)acetic acid, 2-amino-2-(3-fluorophenyl)acetic acid, 2-amino-2-(3-methylphenyl)acetic acid, 2-amino-2-(4ofluorophenyl)acetic acid, 2-amino-2-(4-methylphenyl)acetic acid, 2-amino-2-(4-methoxyphenyl)acetic acid, 2-amino-2-(2-fluorophenyl)acetic acid, 2-amino-2-(2-methylphenyl)acetic acid, 2-amino-2-(4-choromethylphenyl)acetic acid, 2-amino-2-(4-hydroxymethylphenyl)acetic acid, 2-amino-2-[4-(methylthiomethyl)phenyl]acetic acid, 2-amino-2-(4-bromomethylphenyl)acetic acid, 2-amino-2-(4-(methoxymethy)phenyl)acetic acid, 2-amino-2-(4-((N-benzylamino)methyl)phenyl)acetic acid, 2-amino-2-(4-hydroxylphenyl)acetic acid, 2-amino-2-(3-hydroxylphenyl)acetic acid, 2-amino-2-(3-carboxyphenyl)acetic acid, 2-amino-2-(4-aminophenyl)acetic acid, 2-amino-2-(4-azidophenyl)acetic acid, 2-amino-2-(3-t-butyl-4-hydroxyphenyl)acetic acid, 2-amino-2-(3,5-difluoro4-hydroxyphenyl)acetic acid, 2-amino-2-(3,5-dihydroxyphenyl)acetic acid, 2-amino-2-(3-carboxy4-hydroxyphenyl)acetic acid, 2-amino-2-(3,5-di-t-butyl-4-hydroxyphenyl)acetic acid, 2-amino-3-(2-methylphenyl)propanoic acid, 2-amino-3-(4-ethylphenyl)propanoic acid, 2-amino-3-(4-phenylphenyl)propanoic acid, 2-amino-3-(4-benzylphenyl)propanoic acid, 2-amino-3-(3-fluorophenyl)propanoic acid, 2-amino-3-(4-methylphenyl)propanoic acid, 2-amino-3-(4-fluorophenyl)propanoic acid, 2-amino-3-(4-chlorophenyl)propanoic acid, 2-amino-3-(2-chlorophenyl)propanoic acid, 2-amino-3-(4-bromophenyl)propanoic acid, 2-amino-3-(2-bromophenyl)propanoic acid, 2-amino-3-(3-hydroxyphenyl)propanoic acid, 2-amino-3-(2-hydroxyphenyl)propanoic acid, 2-amino-3-(4-mercaptophenyl)propanoic acid, 2-amino-3-(3-trifluoromethylphenyl)propanoic acid, 2-amino-3-(3-hydroxyphenyl)propanoic acid, 2-amino-3-(4-hydroxyphenyl)propanoic acid, 2-amino-3-[4-(hydroxymethy)phenyl]propanoic acid, 2-amino-3-[3-(hydroxymethyl)phenyl]propanoic acid, 2-amino-3-[3-(aminomethyl)phenyl]propanoic acid, 2-amino-3-(3-carboxyphenyl) propanoic acid, 2-amino-3-(4-nitrophenyl)propanoic acid, 2-amino-3-(4-aminophenyl) propanoic acid, 2-amino-3-(4-azidophenyl)propanoic acid, 2-amino-3-(4-cyanophenyl)propanoic acid, 2-amino-3-(4-acetophenyl)propanoic acid, 2-amino-3-(4-guanidinophenyl)propanoic acid, 2-amino-3-[4-(phenylazo)phenyl]propanoic acid, 2-amino-3-[4-(2-phenylethylenyl)phenyl]propanoic acid, 2-amino-3-(4-trialkylsilylphenyl)propanoic acid, 2-amino-3-(2,4-dimethylphenyl)propanoic acid, 2-amino-3-(2,3-dimethylphenyl)propanoic acid, 2-amino-3-(2,5-dimethylphenyl) propanoic acid, 2-amino-3-(3,5-dimethylphenyl)propanoic acid, 2-amino-3-(2,4,6-trimethylphenyl)propanoic acid, 2-amino-3-(3,4,5-trimethylphenyl)propanoic acid, 2-amino-3-(2,3,4,5,6-pentamethylphenyl)propanoic acid, 2-amino-3-(2,4,-difluorophenyl)propanoic acid, 2-amino-3-(3,4,6-difluorophenyl)propanoic acid, 2-amino-3-(2,5,difluorophenyl)propanoic acid, 2-amino-3-(2,6,-difluorophenyl)propanoic acid, 2-amino-3-(2,3,5,6-tetrafluorophenyl)propanoic acid, 2-amino-3-(3,5-dichloro-2,4,6-trifluorophenyl)propanoic acid, 2-amino-3-(2,3-difluorophenyl)propanoic acid, 2-amino-3-(2,3-bistrifluoromethylphenyl)propanoic acid, 2-amino-3-(2,4-bistrifluoromethylphenyl)propanoic acid, 2-amino-3-(2-chloro-5-trifluoromethylphenyl)propanoic acid, 2-amino-3-(2,5-difluorophenyl)propanoic acid, 2-amino-3-(2,3,4,5,6-pentafluorophenyl)propanoic acid, 2-amino-3-(2,3-dibromophenyl)propanoic acid, 2-amino-3-(2,5-dibromophenyl)propanoic acid, 2-amino-3-(3,4-dibromophenyl)propanoic acid, 2-amino-3-(3,4,5-triiodophenyl)propanoic acid, 2-amino-3-(2,3-dihydroxyphenyl)propanoic acid, 2-amino-3-(2,5-dihydroxyphenyl)propanoic acid, 2-amino-3-(2,6-dihydroxyphenyl)propanoic acid, 2-amino-3-(3-bromo-5-methoxyphenyl)propanoic acid, 2-amino-3-(2,5-dimethoxyphenyl)propanoic acid, 2-amino-3-(2,5-dimethoxy4-methylphenyl)propanoic acid, 2-amino-3-(4-bromo-2,5-dimethoxyphenyl)propanoic acid, 2-amino-3-(3-carboxy-4-hydroxyphenyl)propanoic acid, 2-amino-3-(3-carboxy4-aminophenyl)propanoic acid, 2-amino-3-(2-hydroxy-5-nitrophenyl)propanoic acid, 2-amino-3-(2-ethoxy-5-nitrophenyl)propanoic acid, 2-amino-3-(3,4,5-trimethoxyphenyl)propanoic acid, 2-amino-3-(4-azido-2-nitrophenyl)propanoic acid, 2-amino-3-(2-hydroxy-5-nitrophenyl)propanoic acid, 2-amino-3-(2,4-bis-trimethylsilylphenyl)propanoic acid, 2-amino-3-(4-hydroxy-3,5-di-t-butylphenyl)propanoic acid, 2-amino-3-(4-hydroxy-3-benzylphenyl)propanoic acid, 2-amino-3-(4-hydroxy-3-fluorophenyl)propanoic acid, 2-amino-3-(4-hydroxy-2,3,5,6-tetrafluorophenyl)propanoic acid, 2-amino-3-(4-hydroxy-3,5-dichlorophenyl)propanoic acid, 2-amino-3-(4-hydroxy-3-iodophenyl)propanoic acid, 2-amino-3-(4-hydroxy-3,5-diiodophenyl)propanoic acid, 2-amino-3-(4-hydroxy-2-hydroxyphenyl)propanoic acid, 2-amino-3-(4-hydroxy-3-hydroxymethylphenyl)propanoic acid, 2-amino-3-(4-hydroxy-2hydroxy-6-methylphenyl)propanoic acid, 2-amino-3-(4-hydroxy-3-carboxyphenyl)propanoic acid, 2-amino-3-(4-hydroxy-3,5-dinitrophenyl)propanoic acid, substituted thyronines, 2-amino-3-(3,4-dihydroxy-2-chlorophenyl)propanoic acid, 2-amino-3-(3,4-dihydroxy-2-bromophenyl)propanoic acid, 2-amino-3-(3,4-dihydroxy-2-fluorophenyl)propanoic acid, 2-amino-3-(3,4-dihydroxy-2-nitrophenyl)propanoic acid, 2-amino-3-(3,4-dihydroxy-2-methylphenyl)propanoic acid, 2-amino-3-(3,4-dihydroxy-2-ethylphenyl)propanoic acid, 2-amino-3-(3,4-dihydroxy-2-isopropylphenyl)propanoic acid, 2-amino-3-(2-t-butyl-4,5-dihydroxyphenyl)propanoic acid, 2-amino-3-(3-fluoro-4,5-dihydroxyphenyl)propanoic acid, 2-amino-3-(2-fluoro-4,5-dihydroxyphenyl)propanoic acid, 2-amino-3-(2,5,6-trifluoro-3,4-dihydroxyphenyl)propanoic acid, 2-amino-3-(2,6-dibromo-3,4-dihydroxyphenyl)propanoic acid, 2-amino-3-(5,6-dibromo-3,4-dihydroxyphenyl)propanoic acid, 2-amino-3-(2,4,5-trihydroxyphenyl)propanoic acid, 2-amino-3-(2,3,4-trihydroxyphenyl)propanoic acid, 2-amino-3-(3,4-dihydroxy-5-methoxyphenyl)propanoic acid, 2-amino-3-methyl-3-phenylpropanoic acid, 2-amino-3-ethyl-3-phenylpropanoic acid, 2-amino-3-isopropyl-3-phenylpropanoic acid, 2-amino-3-butyl-3-phenylpropanoic acid, 2-amino-3-benzyl-3-phenylpropanoic acid, 2-amino-3-phenylethyl-3-phenylpropanoic acid, 2-amino-3-(4-chorophenyl)-3-phenylpropanoic acid, 2-amino-3-(4-methoxyphenyl)-3-phenylpropanoic acid, 2-amino-3,3-diphenylpropanoic acid, 2-amino-3-[4-(N,N-diethylamino)phenyl]heptanoic acid, 2-amino-3-[4-(N,N-diethylamino)phenyl]pentanoic acid, 2-amino-3-(3,4-dimethoxyphenyl)pentanoic acid, 2-amino-3-(3,4-dihydroxyphenyl)pentanoic acid, 2-amino-3-methyl-3-phenylbutanoic acid, 2-amino-3-ethyl-3-phenylpentanoic acid, 2-amino-3-methyl-3-phenylpentanoic acid, 2-amino-3,3-diphenylbutanoic acid, 2-amino-3-fluoro-3-phenylpropanoic acid, 2-amino-3-methylene-3-phenylpropanoic acid, 2-amino-3-methylmercapto-3-phenylpropanoic acid, 2-amino-4-methylmercapto-4-phenylbutanoic acid, 2-amino-4-(3,4-dihydroxyphenyl)butanoic acid, 2-amino-5-(4-methoxyphenyl)pentanoic acid, 2-amino-4-phenylbutanoic acid, 2-amino-5-phenylpentanoic acid, 2-amino-3,3-dimethyl-5-phenylpentanoic acid, 2-amino-4-phenyl-3-butenoic acid, 2-amino-4-phenoxybutanoic acid, 2-amino-5-phenoxypentanoic acid, 2-amino-2-(indanyl)acetic acid, 2-amino-2-(1-tetralyl)acetic acid, 2-amino-4,4-diphenylbutanoic acid, 2-amino-2-(2-naphthyl)acetic acid, 2-amino-3-(1-naphthyl)propanoic acid, 2-amino-3-(1-naphthyl)pentanoic acid, 2-amino-3-(2-naphthyl)propanoic acid, 2-amino-3-(1-chloro-2-naphthyl)propanoic acid, 2-amino-3-(1-bromo-2-naphthyl)propanoic acid, 2-amino-3-(4-hydroxy-1-naphthyl)propanoic acid, 2-amino-3-(4-methoxy-1-naphthyl)propanoic acid, 2-amino-3-(4-hydroxy-2-chloro-1-naphthyl)propanoic acid, 2-amino-3-(2-chloro4-methoxy-1-naphthyl)propanoic acid, 2-amino-2-(2-anthryl)acetic acid, 2-amino-3-(9-anthryl)propanoic acid, 2-amino-3-(2-fluorenyl)propanoic acid, 2-amino-3-(4-fluorenyl)propanoic acid, 2-amino-3-(carboranyl)propanoic acid, 3-methylproline, 4-methylproline, 5-methylproline, 4,4-dimethylproline, 4-fluoroproline,-4,4-difluoroproline, 4-bromoproline, 4-chloroproline, 4-aminoproline, 3,4-dehydroproline, 4-methylproline, 4-methyleneproline, 4-mercaptoproline, 4-(4-methoxybenzylmercapto)proline, 4-hydroxymethylproline, 3-hydroxyproline, 3-hydroxy-5-methylproline, 3,4-dihydroxyproline, 3-phenoxyproline, 2-aminoproline, 5-aminoproline, 3-carbamylalkylproline, 4-cyano-5-methyl-5-carboxyproline, 4,5-dicarboxyl-5-methylproline, 2-aziridinecarboxylic acid, 2-azetidinecarboxylic acid, 4-methyl-2-azetidinecarboxylic acid, pipecolic acid, 1,2,3,6-tetrahydropicolinic acid, 3,4-methyleneproline, 2.4-methyleneproline, 4-aminopipecolic acid, 5-hydroxypipecolic acid, 4,5-dihydroxypipecolic acid, 5,6-dihydroxy-2,3-dihydroindole-2-carboxylic acid, 1,2,3,4-tetrahydroquinoline-2-carboxylic acid, 6,7-dihydroxy-1,2,3,4-tetrahydroisoquinoline-3-carboxylic acid, 6-hydroxy-1-methyl-1,2,3,4-tetrahydroisoquinoline-3-carboxylic acid, 6,7-dihydroxy-1-methyl-1,2,3,4-tetrahydroisoquinoline-3-carboxylic acid, 1,3-oxazolidine-4-carboxylic acid, 1,2-oxazolidine-3-carboxylic acid, perhydro-1,4-thiazine-3-carboxylic acid, 2,2-dimethylthiazolidine-4-carboxylic acid, perhydro-1,3-thiazine-2-carboxylic acid, selenazolidine4-carboxylic acid, 2-phenylthiazolidine4-carboxylic acid, 2-(4-carboxylicyl)thiazolidine-4-carboxylic acid, 1,2,3,4,4a,9a-hexahydro-beta-carboline-3-carboxylic acid, 2,3,3a,8-atetrahydropyrrolo(2,3b)indole-2-carboxylic acid, 2-amino-3-(2-pyridyl)propanoic acid, 2-amino-3-(3-pyridyl)propanoic acid, 2-amino-3-(4-pyridyl)propanoic acid, 2-amino-3-(2-bromo-3-pyridyl)propanoic acid, 2-amino-3-(2-bromo4-pyridyl)propanoic acid, 2-amino-3-(2-bromo-5-pyridyl)propanoic acid, 2-amino-3-(2-bromo-6-pyridyl)propanoic acid, 2-amino-3-(2-chloro-3-pyridyl)propanoic acid, 2-amino-3-(2-chloro4-pyridyl)propanoic acid, 2-amino-3-(2-chloro-5-pyridyl)propanoic acid, 2-amino-3-(2-chloro-6-pyridyl)propanoic acid, 2-amino-3-(2-fluoro-3-pyridyl)propanoic acid, 2-amino-3-(2-fluoro4-pyridyl)loropanoic acid, 2-amino-3-(2-fluoro-5-pyridyl)propanoic acid, 2-amino-3-(2-fluoro-6-pyridyl)proloanoic acid, 2-amino-3-(1,2-dihydro-2-oxo-3-pyridyl)propanoic acid, 2-amino-3-(1,2-dihydro-2-oxo-4-pyridyl)propanoic acid, 2-amino-3-(1,2-dihydro-2-oxo-5-pyridyl)propanoic acid, 2-amino-3-(1,2-dihydro-2-oxo-6-pyridyl)propanoic acid, 2-amino-3-(5-hydroxy-2-pyridyl)propanoic acid, 2-amino-3-(5-hydroxy-6-iodo-2-pyridyl)propanoic acid, 2-amino-3-(3-hydroxy-4-oxo-1,4dihydro-1-pyridyl)propanoic acid, N-(5-caroxyl-5-aminopentyl)pyridinium chloride, 1,2,5-trimethyl4-(2-amino-2-carboxy-1-hydroxyethyl)pyridinium chloride, 2-amino-2-(5-chloro-2-pyridyl)acetic acid, N-(3-amino-3-carboxypropyl)pyridinium chloride, 2-amino-3-(2-pyrryl)propanoic acid, 2-amino-3-(1-pyrryl)propanoic acid, 2-amino-4-(1-pyrryl)butanoic acid, 2-amino-5-(1-pyrryl)pentanoic acid, 2-amino-3-(5-imidazolyl)-3-methylpropanoic acid, 2-amino-3-(5-imidazolyl)-3-ethylpropanoic acid, 2-amino-3-hexyl-3-(5-imidazolyl)propanoic acid, 2-amino-3-hydroxy-3-(5-irnidazolyl)propanoic acid, 2-amino-3-(4-nitro-5-imidazolyl)proloanoic acid, 2-amino-3-(4-methyl-5-imidazolyl)propanoic acid, 2-amino-3-(2-methyl-5-imidazolyl)propanoic acid, 2-amino-3-(4-fluoro-5-imidazolyl)propanoic acid, 2-amino-3-(2-fluoro-5-imidazolyl)propanoic acid, 2-amino-3-(2-amino-5-imidazolyl)propanoic acid, 2-amino-3-(2-phenylaza-5-imidazolyl)propanoic acid, 2-amino-3-(1-methyl-2-nitro-5-imidazolyl)propanoic acid, 2-amino-3-(1-methyl-4-nitro-5-imidazolyl)propanoic acid, 2-amino-3-(1-methyl-5-nitro-5-imidazolyl)propanoic acid, 2-amino-3-(2-mercapto-5-imidazolyl)propanoic acid, 2-amino-4-(5-imidazolyl)butanoic acid, 2-amino-3-(1-imidazolyl)propanoic acid, 2-amino-3-(2-imidazolyl)propanoic acid, 2-amino-(1-pyrazolyl)propanoic acid, 2-amino-(3-pyrazolyl)propanoic acid, 2-amino-(3,5-dialkyl-4-pyrazolyl)propanoic acid, 2-amino-3-(3-amino-1,2,4-triazol-1-yl)propanoic acid, 2-amino-3-(tetrazol-5-yl)propanoic acid, 2-amino-4-(5-tetrazolyl)butanoic acid, 2-amino-3-(6-methyl-3-indolyl)propanoic acid, 2-amino-3-(4-fluoro-3-indolyl)propanoic acid, 2-amino-3-(5-fluoro-3-indolyl)propanoic acid, 2-amino-3-(6-fluoro-3-indolyl)propanoic acid, 2-amino-3-(4,6,6,7-tetrafluoro-3-indolyl)propanoic acid, 2-amino-3-(-chloro-3-indolyl)propanoic acid, 2-amino-3-(6-chloro-3-indoly)propanoic acid, 2-amino-3-(7-chloro-3-indolyl)propanoic acid, 2-amino-3-(6-bromo-3-indolyl)propanoic acid, 2-amino-3-(7-bromo-3-indolyl)propanoic acid, 2-amino-3-(2-hydroxy-3-indolyl)propanoic acid, 2-amino-3-(7-hydroxy-3-indolyl)propanoic acid, 2-amino-3-(7-hydroxy-3-indolyl)propanoic acid, 2-amino-3-(2-alkylmercapto-3-indolyl)propanoic acid, 2-amino-3-(7-amino-3-indolyl)propanoic acid, 2-amino-3-(4-nitro-3-indolyl)propanoic acid, 2-amino-3-(7-nitro-3-indolyl)propanoic acid, 2-amino-3-(4-carboxy-3-indolyl)propanoic acid, 2-amino-3-(3-indolyl)butanoic acid, 2-amino-3-(2,3-dihydro-3-indolyl)propanoic acid, 2-amino-3-(2,3-dibydro-2-oxo-3-indolyl)propanoic acid, 2-amino-3-alkylmercapto-3-(3-indolyl)propanoic acid, 2-amino-3-(4-aza-3-indolyl)propanoic acid, 2-amino-3-(7-aza-3-indolyl)propanoic acid, 2-amino-3-(7-aza-6-chloro4-methyl-3-indolyl)propanoic acid, 2-amino-3-(2,3-dihydrobenzofuiran-3-yl)propanoic acid, 2-amino-3-(3-methyl-5-7-dialkylbenzofuran-2-yl)propanoic acid, 2-amino-3-(benzothiophen-3-yl)propanoic acid, 2-amino-3-(5-hydroxybenzothiophen-3-yl)propanoic acid, 2-amino-3-eoenzoselenol-3yl)propanoic acid, 2-amino-3-quinolylpropanoic acid, 2-amino-3-(8-hydroxy-5-quinolyl)propanoic acid, 2-amino-2-(5,6,7,8-tetrahydroquinol-5-yl)acetic acid, 2-amino-3-(3-coumarinyl)propanoic acid, 2-amino-2-(benzisoxazol-3-yl)acetic acid, 2-amino-2-(5-methylbenzisoxazol-3-yl)acetic acid, 2-amino-2-(6-methylbenzisoxazol-3-yl)acetic acid, 2-amino-2-(7-methylbenzisoxazol-3-yl)acetic acid, 2-amino-2-(5-bromobenzisoxazol-3-yl)acetic acid, 2-amino-3-(benzimidazol-2-yl)propanoic acid, 2-amino-3-(5,6-dichlorobenzimidazol-2-yl)propanoic acid, 2-amino-3-(5,6-dimethylbenzimidazol-2-yl)propanoic acid, 2-amino-3-(4,5,6,7-hydrobenzimidazol-2-yl)propanoic acid, 2-amino-2-(benzimidazol-5-yl)acetic acid, 2-amino-2-(1,3-dihydro-2,2-dioxoisobenzothiophen-5-yl)acetic acid, 2-amino-2-(1,3-dihydro-2,2-dioxo-2,1,3-benzothiadiazol-5-yl)acetic acid, 2-amino-2-(2-oxobenzimidazol-5-yl)acetic acid, 2-amino-3-(4-hydroxybenzothiazol-6-yl)propanoic acid, 2-amino-3-(benzoxazol-2-yl)propanoic acid, 2-amino-3-(benzothiazol-2-yl)propanoic acid, 2-amino-3-(9-adeninyl)propanoic acid, 2-amino-2-(6-chloro-9-purinyl)acetic acid, 2-amino-2-(6-amino-9-purinyl)acetic acid, 2-amino-3-(6-purinyl)propanoic acid, 2-amino-3-(8-theobromninyl)propanoic acid, 2-amino-2-(1-uracilyl)acetic acid, 2-amino-2-(1-cytosinyl)acetic acid, 2-amino-3-(1-uracilyl)propanoic acid, 2-amino-3-(1-cytosinyl)propanoic acid, 2-amino-4-(1-pyrimidinyl)butanoic acid, 2-amino-4-(4-amino-1-pyrimidinyl)butanoic acid, 2-amino-4-(4-hydroxy-1-pyrimidinyl)butanoic acid, 2-amino-5-(1-pyrimidinyl)pentanoic acid, 2-amino-5-(4-amino-1-pyrimidinyl)pentanoic acid, 2-amino-5-(4-hydroxy-1-pyrimidinyl)pentanoic acid, 2-amino-3-(5-pyrimidinyl)propanoic acid, 2-amino-3-(6-uracilyl)propanoic acid, 2-amino-3-(2-pyrimnidinyl)propanoic acid, 2-amino-3-(6-amino-4-chloro-2-pyrimidinyl)propanoic acid, 2-amino-3-(4-hydroxy-2-pyrimidinyl)propanoic acid, 2-amino-3-(2-amino-4-pyrimidinyl)propanoic acid, 2-amino-3-(4,5-dihydroxypyrimidin-2-yl)propanoic acid, 2-amino-3-(2-thiouracil-6-yl)propanoic acid, 2-amino-2-(5-alkyl-2-tetrahydrofuryl)acetic acid, 2-amino-2-(5-methyl-2,5-dihydro-2-furyl)acetic acid, 2-amino-2-(5-alkyl-2-furyl)acetic acid, 2-amino-2-(2-furyl)acetic acid, 2-amino-2-(3-hydroxy-5-methyl-4-isoxazolyl)acetic acid, 2-amino-3-(4-bromo-3-hydroxy-5-isoxazolyl)propanoic acid, 2-amino-3-(4-methyl-3-hydroxy-5-isoxazolyl)propanoic acid, 2-amino-3-(3-hydroxy-5-isoxazolyl)propanoic acid, 2-amino-2-(3-chloro-D2-isoxazolin-5-yl)acetic acid, 2-amino-2-(3-oxo-5-isoxazolidinyl)acetic acid, 2-amino-3-(3,5-dioxo-1,2,4-oxadiazolin-2-yl)propanoic acid, 2-amino-3-(3-phenyl-5-isoxazolyl)propanoic acid, 2-amino-3-[3-(4-hydroxyphenyl)-1,2,4-oxadiazol-5-yl]propanoic acid, 2-amino-3-(2-thienyl)propanoic acid, 2-amino-2-(2-furyl)acetic acid, 2-amino-2-(2-thienyl)acetic acid, 2-amino-2-(2-thiazolyl)acetic acid, 2-amino-3-(2-thiazolyl)propanoic acid, 2-amino-4-(4-carboxy-2-thiazolyl)butanoic acid, 2-amino-3-(4-thiazolyl)propanoic acid, 2-amino-3-(2-selenolyl)propanoic acid, 2-amino-3-(2-amino-4-selenolyl)propanoic acid, and 2-amino-3-(beta-ribofuranosyl)propanoic acid.

“Amino acids residue” has its customary meaning in the art and refers to an amino acid that is part of a peptide or polypeptide chain; “amino acid residue” as used herein also refers to various amino acids where sidechain functional groups are coupled with appropriate protecting groups known to those skilled in the art. “The Peptides”, Vol 3, 3-88 (1981) discloses numerous suitable protecting groups. Examples of amino acids where sidechain functional groups are coupled with appropriate protecting groups include, but are not limited to, Asp(OMe), Glu(OMe), Hyp(OMe), Asp(O′Bu), Glu(O′Bu), Hyp(O′Bu), Thr(O′Bu), Asp(OBzl), Glu(OBzl), Hyp(OBzl), and Thr(OBzl).

Many TGF-β mimics are composed of, or include, a peptide. The term “peptide” as used herein refers to polymers of amino acids under 100 amino acids in length; in preferred embodiments, less than 30 amino acids in length. A peptide may be more than about 1, 2, 3, 4, 5, 6, 7, 8, 9, or 10 amino acids in length. A peptide may be less than 100, 90, 80, 70, 60, 50, 40, 35, 30, 25, 20, 15, 14, 13, 12, 11, 10, 9, 8, 7, 6, 5, 4, or 3 amino acids in length. Preferred peptides are less than 30 amino acids in length. The polymer may be linear or branched, it may comprise amino acids of any type as defined above, and it may be interrupted by non-amino acids (e.g., peptidomimetics). The term also encompasses an amino acid polymer that has been modified naturally or by intervention; for example, disulfide bond formation, glycosylation, lipidation, acetylation, carboxylation, phosphorylation, ubiquitination, pegylation or any suitable other manipulation or modification, such as conjugation with a labeling component.

The hydrophobicity, charge, ability to form hydrogen bonds, and other properties of amino acids can be involved in producing peptides that act as TGF-β mimics. Of the twenty common amino acids, those with hydrophobic R groups include: Alanine, Valine, Leucine, Isoleucine, Proline, Phenylalanine, Tryptophan and Methionine. Amino acids with uncharged polar R groups include: Glycine, Serine, Threonine, Cysteine, Tyrosine, Asparagine and Glutamine. Amino acids with charged polar R groups include: Aspartic acid and Glutamic acid. Basic amino acids include: Lysine, Arginine and Histidine (at pH 6.0). Amino acids with phenyl groups include: Phenylalanine, Tryptophan and Tyrosine. As will be apparent to those of skill in the art, many unnatural or artificial amino acids also fall within the various types of amino acids, and may be used in embodiments of the invention.

In exemplary peptides useful in the invention, the appropriate functional groups are represented, in many cases, within the following amino acid sequence, which may be the entire TGF-β mimic or may be included in a larger structure: AAᵢ-AAᵢ₊₁-AAᵢ₊₂ . . . -AAᵢ₊ₙ. In some embodiments, TGF-β mimics useful in the methods of the invention contain:

(1) a hydrophobic or neutral amino acid at position i;

(2) a branched hydrophobic at position i+1 (e.g., Val, Ile);

(3) a small aliphatic at position i+2 (e.g., Ala), where positions i+1 and i+2 together form a structure found in TGF-β mimics, the U-bend (β-bend) structure; and

(4) a side-chain containing a hydrogen-bond acceptor shortly thereafter (at position AAᵢ₊ₙ).

For example, in some embodiments, AAᵢ is alanine, asparagine, or leucine, AA₁₊₁ is valine or isoleucine, and AAᵢ₊₂ is alanine. One aspect of TGF-β mimics useful in the invention is the relative positioning of the side-chains of AAᵢ₊₁ and AAᵢ₊₂. The correct positioning of these amino acids can be achieved if AAᵢ₊₁ and AAᵢ₊₂ are in either of two backbone conformations: a β-bend or an “extended-bend” conformation with the backbone (Φ, ψ) angles of AAᵢ₊₁ equal to approximately (−60, +135) and those of AAᵢ₊₂ equal to approximately (−60, −45).

This sequence is often either immediately followed by or has proximal thereto an amino acid with a hydrogen bond acceptor-containing side-chain. Because the amino acid residue with a hydrogen bond acceptor-containing side-chain does not necessarily have to be immediately followed by, that is adjacent to AAᵢ₊₂, it is referred to as AAᵢ₊ₙ where n is an integer equal to or greater than three. If n is greater than 3, then n−3 (“n minus 3”) amino acid residues would be between AAᵢ₊₂ and AAᵢ₊ₙ in the peptide sequence.

For example, the TGF-β mimics may include the sequence AAᵢ-AAᵢ₊₁-AAᵢ₊₂-AAᵢ₊₃ where AAᵢ through AAᵢ₊₂ are as before described and AAᵢ₊₂ is an amino acid residue with a hydrogen bond acceptor-containing side-chain (and is glutamic acid, aspartic acid, glutamine, or asparagine). The original peptide with TGF-β activity and an especially preferred TGF-β mimic for use in the methods of the present invention, cytomodulin, A-N-V-A-E-N-A (SEQ ID NO: 1) is of this class. Here, AAᵢ-AAᵢ₊₁-AAᵢ₊₂-AAᵢ₊₃ corresponds to the second through fifth residues from the N-terminus of cytomodulin, -N-V-A-E-. Other preferred embodiments are the peptides, L-I-A-E-A-K (SEQ ID NO:2) and L-I-A-E-A-A (SEQ ID NO: 11). In these examples, AAᵢ-AAᵢ₊₁-AAᵢ₊₂-AAᵢ₊₃ corresponds to the first four residues of the peptide, L-I-A-E-.

Another example is the sequence AAᵢ-AAᵢ₊₁-AAᵢ₊₂-AAᵢ₊₃-AAᵢ₊₄ where the residue with the hydrogen bond acceptor-containing side-chain is not immediately adjacent, but instead is proximal to, the initial sequence. In this case, AAᵢ through AAᵢ₊₂ are as before, AAᵢ₊₃ is any suitable amino acid, and AAᵢ₊₄ may be glutamic acid, aspartic acid, glutamine, or asparagine. A preferred embodiment of this class is L-I-A-G-E-G (SEQ ID NO: 14). An especially preferred embodiment is the peptide, L-I-A-P-E-A (SEQ ID NO:3). In both examples, the first five N-terminal amino acids correspond to AAᵢ-AAᵢ₊₁-AAᵢ₊₂-AAᵢ₊₃-AAᵢ₊₄.

Yet another example is the sequence AAᵢ-AAᵢ₊₁-AAᵢ₊₂-AAᵢ₊₃-AAᵢ₊₄-AAᵢ₊₅. Here, AAᵢ through AAᵢ₊₂ are as before, AAᵢ₊₃ and AAᵢ₊₄ are suitable amino acids, and AAᵢ₊₅ may be glutamic acid, aspartic acid, glutamine, or asparagine. An especially preferred member of this series is the peptide, L-I-A-G-G-E (SEQ ID NO: 13). In this particular example there is a one to one correspondence between SEQ ID NO:3 and the sequence AAᵢ-AAᵢ₊₁-AAᵢ₊₂-AAᵢ₊₃-AAᵢ₊₄-AAᵢ₊₅.

As discussed above, the original peptide discovered to have TGF-β activity has been named “cytomodulin” and has the sequence A-N-V-A-E-N-A (SEQ ID NO: 1). Cytomodulin when added to cells in culture in the concentration range 10⁻⁹ to 10⁻⁶ M (1.4 pg/mL to 1400 pg/mL), elicits certain highly specific TGF-β effects in several different cell types. For example, among the effects observed is the inhibition of DNA synthesis in Mv-1-Lu mink lung epithelial cells, the growth and colony formation by NRK-49 F fibroblasts in soft agar, and the induction of increased expression of type I collagen in primary cultures of neo-natal human dermal fibroblasts. Moreover, results with human osteogenic sarcoma (HOS) cell line indicate that cytomodulin also may be a mimic for other members of the TGF-β superfamily, such as bone morphogenic proteins (BMPs) and osteogenic protein (OPs), as evidenced by its ability to specifically stimulate markers (alkaline phosphatase and osteonectin) characteristic of the osteoblast phenotype.

Other exemplary TGF-β mimics useful in the methods of the invention are described in U.S. Pat. No. 6,638,912 and are also described in the Examples.

Peptide TGF-β mimics useful in the invention include peptides having at least 70%, at least 80%, at least 90%, at least 95% or greater than 95% sequence identity to the amino acid sequences disclosed herein. In some embodiments, the invention provides methods and compositions that utilize a peptide having at least 70%, at least 80%, at least 90%, at least 95% or greater than 95% sequence identity to an amino acid sequence comprising one of the amino acid sequences of SEQ ID NOS: 1-42. In some embodiments, the invention provides methods and compositions that utilize a peptide having at least 70%, at least 80%, at least 90%, at least 95% or greater than 95% sequence identity to an amino acid sequence comprising one of the amino acid sequences of SEQ ID NOS: 1, 2, 3, 11, 13, or 14. In some embodiments, the invention provides methods and compositions that utilize a peptide having at least 70%, at least 80%, at least 90%, at least 95% or greater than 95% sequence identity to the amino acid sequence of SEQ ID NO: 1.

Percent sequence identity is determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (ibid.). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative MMP-1 variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, SIAM J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

The present invention also includes peptides having a conservative amino acid change, compared with an amino acid sequence disclosed herein. Many such changes have been described specifically. More generally, for example, variants can be obtained that contain one or more amino acid substitutions of SEQ ID NO: 1-42, in which an alkyl amino acid is substituted for an alkyl amino acid in a TGF-β mimic peptide amino acid sequence, an aromatic amino acid is substituted for an aromatic amino acid in a TGF-β mimic peptide amino acid sequence, a sulfur-containing amino acid is substituted for a sulfur-containing amino acid in a TGF-β mimic peptide amino acid sequence, a hydroxy-containing amino acid is substituted for a hydroxy-containing amino acid in a TGF-β mimic peptide amino acid sequence, an acidic amino acid is substituted for an acidic amino acid in a TGF-β mimic peptide amino acid sequence, a basic amino acid is substituted for a basic amino acid in TGF-β mimic peptide amino acid sequence, or a dibasic monocarboxylic amino acid is substituted for a dibasic monocarboxylic amino acid in a TGF-β mimic peptide amino acid sequence. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the invention.

A specialized kind of insertional variant is the fusion protein. It is contemplated that the entire TGF-β mimic peptide or a fragment of the TGF-β mimic peptide may be used to construct a fusion protein to enhance tissue specific or cell specific functions of the TGF-β mimic peptide useful in the invention.

A fusion protein generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of an immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes such as a hydrolase, glycosylation domains, cellular targeting signals or transmembrane regions.

B. Synthesis

For TGF-β mimics which consist of or include peptides, the peptide may be synthesized by any suitable method for producing peptides of a given sequence. Preferably, peptides of the present invention can be synthesized by various suitable methods that are well known in the art, preferably by solid phase synthesis, manual or automated, as first developed by Merrifield and described by Stewart et al. in Solid Phase Peptide Synthesis (1984). Chemical synthesis joins the amino acids in the predetermined sequence starting at the C-terminus. Basic solid phase methods require coupling the C-terminal protected amino acid to a suitable insoluble resin support. Amino acids for synthesis require protection on the alamino group to ensure proper peptide bond formation with the preceding residue (or resin support). Following completion of the condensation reaction at the carboxyl end, the alamino protecting group is removed to allow the addition of the next residue. Several classes of α-protecting groups have been described, see Stewart et al. in Solid Phase Peptide Synthesis (1984), with the acid labile, urethane-based tertiary-butyloxycarbonyl (Boc) being the historically preferred. Other protecting groups, and the related chemical strategies, may be used, including the base labile 9-fluorenylmethyloxycarbonyl (FMOC). Also, the reactive amino acid sidechain fumctional groups require blocking until the synthesis is completed. The complex array of functional blocking groups, along with strategies and limitations to their use, have been reviewed by Bodansky in Peptide Synthesis (1976) and, Stewart et al. in Solid Phase Peptide Synthesis (1984).

Solid phase synthesis is initiated by the coupling of the described C-terminal α-protected amino acid residue. Coupling requires activating agents, such as dicyclohexycarbodiimide (DCC) with or without 1-hydroxybenzotriazole (HOBT), diisopropylcarbodiimide (DIIPC), or ethyldimethylaminopropylcarbodiimide (EDC). After coupling the C-terminal residue, the α-amino protected group is removed by trifluoroacetic acid (25% or greater) in dichloromethane in the case of acid labile tertiary-butyloxycarbonyl (Boc) groups. A neutralizing step with triethylamine (10%) in dichloro-methane recovers the free amine (versus the salt). After the C-terminal residue is added to the resin, the cycle of deprotection, neutralization and coupling, with intermediate wash steps, is repeated in order to extend the protected peptide chain. Each protected amino acid is introduced in excess (three to five fold) with equimolar amounts of coupling reagent in suitable solvent. Finally, after the completely blocked peptide is assembled on the resin support, reagents are applied to cleave the peptide form the resin and to remove the side chain blocking groups. Anhydrous hydrogen fluoride (HF) cleaves the acid labile tertiary-butyloxycarbonyl (Boc) chemistry groups. Several nucleophilic scavengers, such as dimethylsulfide and anisole, are included to avoid side reactions especially on side chain functional groups.

Slight amino acid modifications to a peptide TGF-β mimic sequence will not affect the peptide's ability to form suitable TGF-β mimics. These modifications include techniques to confer resistance to enzymatic degradation such as adding blocking groups to both the N- and C-terminal residues. Another method for preventing degradation and premature clearance by the renal system is the use of unnatural amino acid substitutes in the peptide sequence. For example, N-methyl-alanine is often substituted for alanine and α-amino isobutryic acid and α-amino butyric acid are substitutes for bulky hydrophobic amino acids.

Recombinant techniques, as known in the art, may also be used to produce peptides suitable for the methods of the invention. Naturally-occurring proteins may be cleaved to produce a desired TGF-β mimic. Methods of designing and screening small molecules may also be used. Methods to generate and screen peptidomimetics may also be useful in producing TGF-β mimics. Substances that are a mixture of peptide and peptidomimetics may also be used.

II. Methods of Treatment

The methods of the invention have wide applicability to the treatment or prophylaxis of skin conditions, to soft tissue augmentation, e.g., cosmetic augmentation and augmentation of sphincters, and to enhancement of wound healing. The methods include administering to an individual an effective amount of a TGF-β mimic; in the case of soft tissue augmentation, the TGF-β mimic is generally administered in combination with a matrix. The mode of administration and dosage regimens will vary depending on the skin condition or wound which is to be treated or the type of tissue augmentation to be achieved. Generally, for wound healing or skin conditions, topical administration will be preferred. For soft tissue augmentation, injection or implantation of a matrix containing TGF-β mimic is generally the preferred method.

The present invention provides methods, pharmaceutical compositions, and kits for the treatment of individuals. The term “individual” as used herein includes humans as well as other mammals. The term “treating” as used herein includes achieving a therapeutic benefit and/or a prophylactic benefit. By therapeutic benefit is meant eradication or amelioration of the underlying disorder or condition being treated. For example, in a patient with dermatitis, therapeutic benefit includes eradication or amelioration of the underlying dermatitis. As another example, in a patient with a wound, therapeutic benefit includes accelerated wound healing, healing with less pain or disability, and/or healing with reduced scarring. Also, a therapeutic benefit is achieved with the eradication or amelioration of one or more of the physiological or psychological symptoms associated with the underlying condition such that an improvement is observed in the patient, notwithstanding the fact that the patient may still be affected by the condition. For example, a TGF-β mimic provides therapeutic benefit not only when a skin condition is eradicated, but also when an improvement is observed in the individual with respect to the skin condition and its attendant consequences, such as psychological consequences. Similarly, TGF-β mimics can provide therapeutic benefit in ameliorating conditions requiring soft tissue augmentation, such as by increasing functioning of a sphincter, e.g., a reduction in incontinence with augmentation of a urinary sphincter, even if the condition is not completely eradicated. A prophylactic benefit of treatment includes prevention of a condition, retarding the progress of a condition (e.g., a progressive skin condition), or decreasing the likelihood of occurrence of a condition. As used herein, “treating” or “treatment” includes prophylaxis.

As used herein, the term “effective amount” can be an amount sufficient to effect beneficial or desired clinical results. An effective amount can be administered in one or more administrations. In terms of treatment, an “effective amount” of a TGF-β mimic of the invention is an amount that is sufficient to palliate, ameliorate, stabilize, reverse or slow the progression of skin condition, or to enhance wound healing, or to provide a sufficient effect so as to provide cosmetic or therapeutic augmentation of a soft tissue. An “effective amount” may be of a TGF-β mimic used alone or in conjunction with one or more agents used to treat a disease or disorder.

A. Treatment of Skin Conditions

The skin is subject to a number of conditions that result in alterations of function and/or appearance that are considered undesirable, either from a health standpoint or cosmetically, or both. Such conditions can lead to physiological discomfort or harm and/or psychological discomfort. Thus, as used herein, “skin conditions” (also referred to herein as “dermatological” or “cutaneous” conditions) are conditions that result in an abnormality or decrease in function or appearance of the skin, generally leading to physiological or psychological discomfort, and in some cases pathological consequences.

Exemplary skin conditions that may be treated by the methods of the invention include, but are not limited to, acne, dermatitis, psoriasis, actinic keratosis, urticaria, rosacea, mucositis, insect bites, hives, basal cell carcinoma, squamous cell carcinoma, keratoacanthoma, papilloma and malignant melanoma.

In some embodiments, the methods of the present invention treat dermatological conditions associated with abnormal proliferation of skin cells, marked by either inflammatory or non-inflammatory components. Many common diseases of the skin, such as psoriasis, basal and squamous cell carcinoma, keratoacanthoma and actinic keratosis are characterized by localized abnormal proliferation and growth. For example, in psoriasis, which is characterized by scaly, red, elevated plaques on the skin, the keratinocytes are known to proliferate much more rapidly than normal and to differentiate less completely. These conditions are marked by unwanted or aberrant proliferation of cutaneous tissue, typically characterized by epidermal cell proliferation or incomplete cell differentiation, and include, for example, X-linked ichthyosis, acne, psoriasis, dermatitis, epidermolytic hyperkeratosis, basal cell carcinoma, squamous cell carcinoma, and keratoacanthoma.

In some embodiments, the methods of the invention are used to treat psoriasis. Therapeutic preparations of a TGF-β mimic, e.g., which promote quiescence or differentiation, can be used to treat varying forms of psoriasis, be they cutaneous, mucosal or ungual. The term “psoriasis,” as used herein, refers to a hyperproliferative skin disorder which alters the skin's regulatory mechanisms. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the dermis layer and polymorphonuclear leukocyte infiltration into the epidermis layer resulting in an increase in the basal cell cycle. Additionally, hyperkeratotic and parakeratotic cells are present. Treatment with a TGF-β mimic can be used to reverse the pathological epidermal activation and can provide a basis for sustained remission of the disease.

Treatment may also include other treatments of psoriasis, such as topical treatments with active agents such as anthralin, calcipotriene, coal tar, salicylic acid, steroids, or tazarotene; light treatment such as pulsed laser or excimer, PUVA (UVA in combination with psoralen), sunlight, or UVB treatment; and systemic agents such as cyclosporin, methotrexate, or oral retinoids. Such compositions and treatments and the standard regimens are known in the art.

In some embodiments, the methods of the invention are used to treat acne. “Acne,” as that term is used herein, refers to a multifactorial disease most commonly occurring in teenagers and young adults, and is characterized by the appearance of inflammatory and noninflammatory lesions on the face and upper trunk. The basic defect which gives rise to acne, i.e., acne vulgaris, is hypercornification of the duct of a hyperactive sebaceous gland. Hypercornification blocks the normal mobility of skin and follicle microorganisms, and in so doing, stimulates the release of lipases by Propinobacterium acnes and Staphylococcus epidermidis bacteria and Pitrosporum ovale, a yeast. Treatment with a TGF-β mimic, particularly topical preparations, prevents the transitional features of the ducts, e.g. hypercornification, which lead to lesion formation.

Treatment methods of the invention for acne may further include other anti-acne agents, for example, antibiotics, retinoids and antiandrogens. Forms and dosage regimens are well-known in the art.

The present invention also provides methods for treating various forms of dermatitis. “Dermatitis,” as used herein, is a descriptive term referring to poorly demarcated lesions which are either pruritic, erythematous, scaly, blistered, weeping, fissured or crusted. These lesions arise from any of a wide variety of causes. The most common types of dermatitis are atopic, contact and diaper dermatitis. For instance, seborrheic dermatitis is a chronic, usually pruritic, dermatitis with erythema, dry, moist, or greasy scaling, and yellow crusted patches on various areas, especially the scalp, with exfoliation of an excessive amount of dry scales. Stasis dermatitis is an often chronic, usually eczematous dermatitis. Actinic dermatitis is dermatitis that is due to exposure to actinic radiation such as that from the sun, ultraviolet waves or x- or gamma-radiation. TGF-β mimics can be used in the treatment and/or prevention of certain symptoms of dermatitis caused by unwanted proliferation of epithelial cells.

In some embodiments, treatment of dermatitis according to the methods of the invention includes administration of an effective amount of another active agent, e.g., topical and/or systemic corticosteroids, antipruritics, and antibiotics. Forms and dosage regimens are well-known in the art.

A variety of other keratotic lesions are also candidates for treatment with the methods of the invention. Actinic keratoses, for example, are superficial inflammatory premalignant tumors arising on sun-exposed and irradiated skin. The lesions are erythematous to brown with variable scaling. Current therapies include excisional and cryosurgery. These treatments are painful, however, and often produce cosmetically unacceptable scarring. Accordingly, treatment of keratosis, such as actinic keratosis, can include application, preferably topical, of a TGF-β mimic in amounts sufficient to inhibit hyperproliferation of epidermal/epidermoid cells of the lesion, optionally in conjunction with other active ingredients.

Basal cell carcinoma, squamous cell carcinoma, papilloma, and keratoacanthoma may also be treated by the methods of the invention. The term “carcinoma” refers to a malignant new growth made up of epithelial cells tending to infiltrate surrounding tissues and to give rise to metastases. Exemplary carcinomas include: “basal cell carcinoma”, which is an epithelial tumor of the skin that, while seldom metastasizing, has potentialities for local invasion and destruction, “squamous cell carcinoma”, which refers to carcinomas arising from squamous epithelium and having cuboid cell. “Keratoacanthoma” is a relatively common low-grade malignancy that originates in the pilosebaceous glands and closely and pathologically resembles squamous cell carcinoma. Another carcinomatous epithelial growth is “papillomas”, which refers to benign tumors derived from epitheliurn and having a papillomavirus as a causative agent. All of these carcinomas may be treated by methods of the invention by administering an effective amount of a TGF-β mimic to the affected individual, typically as a topical preparation. Treatment may be in combination with other treatments as are known in the art.

In addition, malignant melanoma may be treated by methods of the invention. TGFβ is known to have at least two receptors, the TGF-β RI receptor and the TGFβ- RII receptor. Melanomas are accompanied by TGFβ-RII defects, and, without being bound by theory, it is thought that TGFβ mimics, e.g., cytomodulin, directly address TGF-β R-I, bypassing TGFβ-RII, and inducing apoptosis in malignant melanoma cells. Methods of the invention include treatment of an individual suffering from malignant melanoma by administering to the individual an effective amount of a TGFβ mimic, alone or in combination with other agents known in the art for the treatment of malignant melanoma.

Other exemplary skin and mucus membrane conditions that may be treated by the methods of the invention include insect bites, hives (urticaria), rosacea, and mucositis.

Insect bites, as used herein, is a general term that includes the bite or sting of any arthropod, including all biting or stinging arthropods, e.g., insects, spiders, ticks, scorpions, and the. like. In these embodiments, a topical formulation comprising a TGF-β mimic may be applied to the affected area at a frequency and dosage effective to ameliorate or eliminate the symptoms of the bite or sting. Other treatments, as known in the art, may be used in combination with the TGF-β mimic.

Urticaria, commonly known as hives, consists of circumscribed areas of raised erythema and edema of the superficial dermis. Urticaria can be acute or chronic, and a variety of urticarial variants exist, such as hereditary angioedema and anaphylaxis. By definition, the acute form of urticaria lasts less than 4-6 weeks, and the chronic form lasts more than 4-6 weeks. The list of causes is extensive. When a patient presents with acute urticaria within 24 hours of onset, a cause often can be determined. However, in many cases of both acute and chronic urticaria, the cause can be difficult or even impossible to determine. The methods of the invention include methods for treating urticaria by administering to the affected individual an effective amount of a TGF-β mimic.

In some embodiments of the invention, the methods of treating urticaria include administering to the affected individual an effective amount of a TGF-β mimic in combination with other treatments known in the art for treating urticaria. Other treatments include, but are not limited to, H1 antihistamine agents, H2 antihistamines may be added, glucocorticosteroids, doxepin (e.g., topical therapy with 5% doxepin cream (Zonalon)), capsaicin, and cyproheptadine.

In some embodiments the invention provides methods of treating rosacea. Rosacea is a chronic disease of the skin of the face whose cause is unknown. It is marked in early stages by frequent flushing of the center of the face, which may include the forehead, nose, cheeks, and chin. The flushing often is accompanied by a burning sensation, particularly when creams or cosmetics are applied to the face. Sometimes the face is swollen slightly. Vascular rosacea causes persistent flushing and redness. Blood vessels under the skin of the face may dilate, showing through the skin as small red lines, a condition known as telangiectasia. The affected skin may be swollen slightly and feel warm. Inflammatory rosacea causes persistent redness and papules (pink bumps) and pustules (bumps containing pus) on the skin. Eye inflammation and sensitivity as well as telangiectasia also may occur. In the most advanced stage of rosacea, the skin becomes a deep shade of red and inflammation of the eye is more apparent. Numerous telangiectases are often present, and nodules in the skin may become painful. A condition called rhinophyma also may develop, which is characterized by an enlarged, bulbous, and red nose resulting from enlargement of the sebaceous (oil-producing) glands beneath the surface of the skin on the nose. People who have rosacea also may develop a thickening of the skin on the forehead, chin, cheeks, or other areas.

The invention provides methods for treatment of rosacea that include administration to an individual suffering from rosacea of an effective amount of a TGF-β mimic. In some embodiments, the TGF-β mimic is administered in combination with other treatments known in the art for treating rosacea. These include the use of topical antibiotic, such as metronidazole, which is applied directly to the affected skin. In more severe cases, and for cases affecting the eyes, an oral antibiotic may be used. Exemplary oral antibiotics include tetracycline, minocycline, erythromycin, and doxycycline. Electrosurgery and laser surgery are treatment options if red lines caused by dilated blood vessels appear in the skin or if rhinophyma develops. For patients with rhinophyma, surgical removal of the excess tissue to reduce the size of the nose can be performed.

In some embodiments the invention provides methods of treating mucositis. Mucositis is swelling, irritation, and ulceration of the mucosal cells that line the digestive tract. Mucositis can occur anywhere along the digestive tract from the mouth to the anus. It can be a side effect of chemotherapy. Methods of the invention include treating an individual suffering from mucositis by administering to the individual an effective amount of a TGF-β mimic, optionally in combination with other treatments known in the art for treating mucositis. If the mucositis is present in the mouth, the TGF-β mimic is administered in a form suitable for topical oral use. Mucositis localized to the anus may be treated by topical non-oral formulas. Mucositis in the rest of the digestive tract may be treated by ingestible forms of TGF-β mimic.

In some embodiments, mucositis is treated by administration of a TGF-β mimic in combination with other methods for treatment of mucositis. The treatment will depend upon the severity of the symptoms and the white cell blood count. Treatments include frequent cleaning of the mouth, use of a water-soluble jelly to help lubricate the mouth, frequent rinsing of the moutn with plain sterile water or a salt water mouthwash, avoiding the use of liquids containing alcohol, including many proprietary mouthwashes, the use of pain relief gels or creams, or the use of painkillers such as paracetamol, or, in severe cases, stronger painkillers either by mouth or injection. Allopurinol and vitamin E may also be used in combination with the TGF-β mimics of the invention.

Also included in conditions which may be treated by the subject method are disorders specific to non-humans, such as mange.

In any of the methods of the invention for treating skin conditions, a single TGF-β mimic may be used, or more than one TGF-β mimic may be used. Any suitable TGF-β mimic may be used in accordance with the description herein. In some embodiments, the TGF-β mimic used is that of SEQ ID NO: 1. In some embodiments, the TGF-β mimic used is that of SEQ ID NO: 2. In some embodiments, the TGF-β mimic used is that of SEQ ID NO: 3. In some embodiments, the TGF-β mimic used is that of SEQ ID NO: 11. In some embodiments, the TGF-β mimic used is that of SEQ ID NO: 13. In some embodiments, the TGF-β mimic used is that of SEQ ID NO: 14. In some embodiments, a TGF-β of any of SEQ ID NOS: 4-10, 12, or 15-42 may be used. In some embodiments, a combination of TGF-β mimics is used.

In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1.

The TGF-β mimics may be administered in pharmaceutically acceptable form, depending on the type of treatment desired, e.g. orally, parenterally, topically, and the like. Types of administration suitable to treatment are well-known in the art. See, e.g., Remington's Pharmaceutical Sciences, Gennaro, A R, ed., 20th edition, 2000: Williams and Willins PA., USA. Concentrations and dosages will be dependent upon the condition being treated, the individual suffering from the condition, its severity, type, and duration, and the judgment of the attending health professional, e.g., physician. Concentrations for non-topical administration can be, e.g., less than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.01, 0.1, 1, 5, or 10% (all concentration percentages given herein are w/w unless otherwise indicated). Further concentrations are given in the discussion of compositions, below.

In some embodiments, TGF-β mimic is administered topically. In these embodiments, any pharmaceutically acceptable carrier, as described in more detail below, may be used. In embodiments of methods of the invention, the concentration of TGF-β mimic used for topical treatment may be more than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.0 1, 0.1, 1, 5, or 10%. In some embodiments, the TGF-β mimic is administered topically at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

In topical administration, skin coverage may also be described in terms of total ng of TGF-β mimic/cm2 of skin; in these terms, a typical coverage per administration would be more than about 3, 6, 60, 600, 6000, 60,000, or 600,000 ng/cm2 of skin; less than about 900,000, 600,000, 60,000, 6000, 600, 60, or 6 ng/cm2 of skin; or about 6 ng/cm2 to about 600 ng/cm2 of skin; or about 60 ng/cm2 of skin.

Topical administration may be by any means that brings the TGF-β mimic and, optionally, other treatment agents, in contact with the surface of the skin, including application as a gel, lotion, cream, liposomal preparation or the like, with or without occlusion, or application as a plaster, patch, mask, glove, or similar device for extended contact with an affected area of skin. For treatment of skin conditions or to produce a desired therapeutic effect, the frequency and duration of administration of a formulation comprising a TGF-β mimic is dependent on factors including the nature of the formulation (e.g., concentration, presence or lack of other treatment agents, vehicle type), the severity and extent of the condition, and in some cases the judgment of a health care professional, e.g., attending physician.

Topical application may be more than about once, twice, three times, four times, five times, or six times per week, or more than once, twice, three times, four times, five times, or six times per day. Frequency of application may be less than about twice, three times, four times, five times, or six times per week, or less than about once, twice, three times, four times, five times, or six times per day. Some embodiments of the invention provide a method for treatment a skin condition in an individual in need of such treatment by topical administration of an effective amount of a TGF-β mimic. In some embodiments, the TGF-β mimic is administered an average of about once per day to once per week; in some embodiments, the TGF-β mimic is administered an average of about once per day; in some embodiments, the TGF-β mimic is administered an average of about once or twice per day; in some embodiments, the TGF-β mimic is administered an average of about once to three times per day; in some embodiments, the TGF-β mimic is administered an average of more than about three times per day. In some embodiments, the TGF-β mimic is administered an average of about twice per day, typically in the morning upon rising and in the evening before retiring. Topical administration may be without a covering. Alternatively, topical administration may include the use of a covering over the TGF-β mimic, which may be occlusive or non-occlusive. For example, administration in the evening before retiring may include covering the administered area with an occlusive or non-occlusive covering, which may remain in place during sleep.

The duration of treatment generally will depend on the response of the skin to therapeutic treatment. Treatment may continue at the discretion of the individual being treated. In some cases, administration or application of a formulation containing a TGF-β mimic may be more frequent at the beginning of treatment and less frequent as treatment continues and the condition is ameliorated or the desired effect is achieved. In some cases, treatment may continue indefinitely in order to maintain a condition in abeyance or in an improved state, to delay onset of a skin condition (e.g., mucositis), or to slow the progression of a skin condition. These modifications of frequency and duration are easily accomplished by the individual being treated or by the attending health care professional, e.g., physician.

Some embodiments of treatment of a skin condition employ topical administration of a lotion, in some embodiments comprising a mixture of emulsifying lanolin alcohols, waxes, and oils (e.g., EUCERIN™) or a mixture of petrolatum or mineral oil, a quaternary ammonium compound, a fatty alcohol, and a fatty ester emollient (e.g., CUREL™, see U.S. Pat. No. 4,389,418), comprising a TGF-β mimic. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). The concentration of the TGF-β mimic (e.g., cytomodulin) can be more than about 0.00005%, 0.0001%; 0.001%; 0.01%; 0.1%; 1%, or 10%; the TGF-β mimic can be at a concentration of less than about 15%, 10%, 1%, 0.1%, 0.01%; 0.001%; or 0.0001%; in some embodiments, about 0.0001% to about 0.01%, in some embodiments about 0.001%. The lotion containing the TGF-β mimic (e.g., cytomodulin) is applied at a frequency of once to three times per day, in some embodiments once per day, until the desired result, e.g., reduction or elimination of the skin condition being treated, is observed, followed by topical application once to five times per week, in some embodiments about once to three times per week, in some embodiments once per week, thereafter.

If the TGF-β mimic is used in combination with another skin treatment method or composition, any combination of the TGF-β mimic and the additional method or composition may be used. Thus, for example, if use of a TGF-β mimic is in combination with another treatment agent, the two may be administered simultaneously, consecutively, in overlapping durations, in similar, the same, or different frequencies, etc. In some cases a composition will be used that contains a TGF-β mimic in combination with one or more other treatment agents.

Other treatment agents that may be used in methods of the invention are described in more detail above, in connection with specific skin conditions. Further treatment agents are also described below, in connection with compositions. Dosages, routes of administration, administration regimes, and the like for these agents are well-known in the art.

B. Soft Tissue Augmentation

The invention also provides methods for soft tissue augmentation in an individual in need of such augmentation, or desiring such augmentation. In some embodiments, soft tissue augmentation is used for cosmetic enhancement, or in conjunction with standard procedures for cosmetic enhancement to accelerate recovery. In some embodiments, soft tissue augmentation is used in reconstructive augmentation. In some embodiments, soft tissue augmentation is used for sphincter enhancement to improve sphincter function. In some embodiments, soft tissue enhancement may be used for, or in conjunction with, periodontal tissue repair, maxillofacial surgery, nasal surgery, or oral surgery.

In these embodiments of the invention, the TGF-β mimic may be provided in a pharmaceutically acceptable carrier, or in a matrix. The matrix may be any suitable matrix, e.g., polymer, that is compatible with injection or implantation in an individual, and may be resorbable or non-resorbable. Examples of suitable polymers may be found in U.S. Pat. Nos. 5,661,127; 5,780,436; and 6,638,912, and references therein.

Augmentation for cosmetic purposes include augmentation of any site that is desired to be augmented; examples include breast augmentation, facial implant, skin and body contour shaping, but any area that is desired to be augmented may be treated by the methods of the invention. In these embodiments, the TGF-β mimic is typically combined with a matrix material, and the combination may be used in the same way as standard injections or implants, or modified as dictated by the clinician's experience. In addition, TGF-β mimics may be used for hair restoration. Reconstructive augmentation includes reconstructive surgery for traumatic wounds, cancer reconstruction, and the like.

Present methods of cosmetic and reconstructive augmentation require the injection or implantation of any of a variety of substances that are subject to migration and other undesirable effects. The methods of the invention utilize initial implantation or injection of the TGF-β mimic, generally in combination with a matrix material. Without being bound by theory, it is believed that the TGF-β mimic stimulates the body to provide its own tissue, comprising cellular and extracellular components (e.g., collagen and elastin) at the site of implantation or injection. Furthermore, if the matrix with which the TGF-β mimic is associated is constructed from biodegradable materials, the matrix eventually is resorbed, and no exogenous materials remain in the body. Thus, the use of the methods of the present invention reduces the risks associated with migration, bursting, and other events that may follow standard augmentation procedures.

Sphincter augmentation may be used to provide bulk in a sphincter that has lost functional ability, typically through loss of muscle mass or function. Such augmentation is usefull in, e.g., urinary, esophageal, and duodenal sphincters to restore functional ability. Present methods of augmentation for sphincters, which generally utilize introduction of inert substances to augment the sphincter muscle and associated structures, suffer many of the same drawbacks as cosmetic augmentation procedures, especially migration of the inserted material. The methods of the present invention provide for augmentation without the high risk of migration; in some embodiments, the TGF-β mimic may be introduced directly in or around the sphincter to be treated and stimulate tissue proliferation; preferably, the TGF-β mimic is introduced in a matrix, as for cosmetic augmentation; most preferably, the matrix is biodegradable and resorbable, so that eventually the tissue that has been added to the area by action of the TGF-β mimic replaces the matrix.

TGF-β mimics may also be used in treatment of periodontal disease, including tissue repair, and in maxillofacial surgery, nasal surgery, and oral surgery, to augment or replace tissue. In some embodiments, methods of the invention can be used to help control guided tissue regeneration, such as when used in conjunction with bioresorptable materials. For example, incorporation of periodontal implants, such as prosthetic teeth, can be facilitated by the methods of the invention. Reattachment of a tooth involves both formation of connective tissue fibers and re-epithelization of the tooth pocket. The methods provided herein can be used to accelerate tissue reattachment by controlling the mitotic function of basal epithelial cells in early stages of wound healing. In addition, cytomodulins promote bone and other connective tissue formation, resulting in accelerated repair.

In any of the methods of the invention for soft tissue augmentation, a single TGF-β mimic may be used, or more than one TGF-β mimic may be used. Any suitable TGF-β mimic may be used in accordance with the description herein. In some embodiments, the TGF-β mimic used is that of SEQ ID NO: 1. In some embodiments, the TGF-β mimic used is that of SEQ ID NO: 2. In some embodiments, the TGF-β mimic used is that of SEQ ID NO: 3. In some embodiments, the TGF-β mimic used is that of SEQ ID NO: 11. In some embodiments, the TGF-β mimic used is that of SEQ ID NO: 13. In some embodiments, the TGF-β mimic used is that of SEQ ID NO: 14. In some embodiments, a TGF-β of any of SEQ ID NOS: 4-10, 12, or 15-42 may be used. In some embodiments, a combination of TGF-β mimics is used.

In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1.

As described herein, the TGF-β mimic is typically administered within a matrix for soft tissue augmentation, which is injected or implanted. In these embodiments, the concentration of TGF-β mimic used for soft tissue augmentation within the matrix material may be more than about 0.00001, 0.00005, 0.0001, 0.001, 0.01, 0.1, 1, 5, 10, 15, 20, 25, 30, 35, or 40%. In some embodiments, the concentration of the TGF-β mimic is more than about 0.00005%. The concentration of TGF-β mimic may be less than about 0.0001, 0.001, 0.001, 0.01, 0.1, 1, 5, 10, 15, 20, 25, 30, 35, 40, 45, 50, 55, or 60% (all concentration percentages given herein are w/w unless otherwise indicated). In some embodiments, the TGF-β mimic is administered within the matrix material at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

Other materials may be used with the implant or injection in order to improve tissue integration. Such materials are known in the art.

C. Enhancement of Wound Healing

The present invention also provides methods to enhance wound healing. In some embodiments, the invention provides a method for enhancing wound healing in an individual suffering from a wound comprising applying an effective amount of a TGF-β mimic, e.g., cytomodulin, to the wound.

The types of wounds that may be treated are not limited in any way. A “wound” is any internal or external bodily injury or lesion. Wounds may be caused, for example, by mechanical, chemical, or thermal means, or as the result of a disease or disorder. A wound typically disrupts the normal continuity of one or more bodily structures. Wounds include, without limitation, wounds in which the skin is unbroken (contusions), wounds in which the skin is broken by a cutting instrument (incisions) and wounds in which the skin is broken by a dull or blunt instrument (lacerations). Wounds may be caused by accidents or by intentional acts such as surgical procedures. Wounds may also result from, or be related to a disease or disorder. For example, the wounds may be related to diabetes or cancer. Non-exclusive examples of wounds include burn wounds, decubitus ulcers, venous-stasis ulcers, neuropathic ulcers, diabetic ulcers, poorly-healing wounds, normal surgical invasions, traumatic wounds, pyogenic granuloma, pyoderma gangrenosum, oral lesions, mucosal lesions, airway/lung lesions, gastric ulcerations, intestinal ulcerations, ulcerative colitis, Crohn's disease and opthalmic ulcerations. In addition, in some embodiments, wounds that may be treated by the present compositions and methods include gastric ulcers, pancreas, liver, kidney, spleen, blood vessel injuries and other internal wounds.

A “chronic wound,” as used herein, refers to a wound that has not healed within 30 days.

“Enhancing wound healing” or “promoting wound healing,” as used herein, refers to modulating wound healing in a desired manner. For example, enhancing or promoting wound healing includes initiating wound healing (e.g., in the case of non-healing chronic wounds), accelerating wound healing, providing more complete healing, allowing healing of a wound with less or no scarring, decreasing secondary wound characteristics such as pain, inflammation, danger of infection, bleeding, odor, and the like.

“Reduced scarring” refers to formation of less scar tissue during wound healing, or to the formation of more organized or less prominent or less visible scar tissue, than would likely occur without treatment. For example, reduced scarring can refer to the formation of a scar of smaller dimensions than would likely occur without treatment; reduced scarring can also refer to formation of a more organized or less prominent scar than would likely occur without treatment, e.g., to formation of a more nearly normal scar in an individual who is usually subject to keloid formation. “Scarless wound healing” or “healing without a scar” refers to healing of a wound substantially without scar that is visible to the naked eye, although microscopic healing or healing beneath the skin surface might still involve some degree of scarring.

The term “dressing” refers broadly to any suitable material applied to a wound for protection, absorbance, drainage, to provide therapeutic substances to the wound, and the like. Numerous types of dressings are commercially available, including, but not limited to, films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer)

The present invention encompasses compositions and methods for enhancing wound healing by the use of a TGF-β mimic. The compositions and methods of the invention are also useiful in enhancing the repair of other types of tissue damage, e.g., traumatic or congenital, wherein the repair and/or regeneration of tissue defects or damage is desired.

In particular, in some embodiments the methods of the invention result in healing of a wound with reduced, and, in some embodiments, no visible scar formation. In some embodiments, the TGF-β mimic is applied in combination with one or more other components, such as antibiotics, analgesics, anesthetics, and the like. In some embodiments, the wound is a surgical wound. In some embodiments, the wound is a traumatic wound. In some embodiments, the individual is a patient with diabetes. In some embodiments, the individual is a burn patient. In some embodiments, the wound is a chronic wound.

Thus, in some embodiments, the invention provides methods of enhancing the healing of a wound by administering to an individual with a wound an effective amount of a TGF-β mimic. As described, the wound may be of any type. Non-exclusive examples of wounds that may be treated by the methods of the invention include traumatic wounds, surgical wounds, burn wounds, and chronic wounds. Wounds include wounds to deep tissue and/or organs, such as may occur in traumatic or surgical wounds.

In any of the methods of the invention for enhancing wound healing, a single TGF-β mimic may be used, or more than one TGF-β mimic may be used. Any suitable TGF-β mimic may be used in accordance with the description herein. In some embodiments, the TGF-β mimic used is that of SEQ ID NO: 1. In some embodiments, the TGF-β mimic used is that of SEQ ID NO: 2. In some embodiments, the TGF-β mimic used is that of SEQ ID NO: 3. In some embodiments, the TGF-β mimic used is that of SEQ ID NO: 1. In some embodiments, the TGF-β mimic used is that of SEQ ID NO: 13. In some embodiments, the TGF-β mimic used is that of SEQ ID NO: 14. In some embodiments, a TGF-β mimic of any of SEQ ID NOS: 4-10, 12, or 15-42 may be used. In some embodiments, a combination of TGF-β mimics is used.

In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1.

The TGF-β mimics may be administered in pharmaceutically acceptable form, depending on the type of treatment desired, e.g., orally, parenterally, topically, and the like. Types of administration suitable to treatment are well-known in the art. See, e.g., Remington's Pharmaceutical Sciences, Gennaro, A R, ed., 20th edition, 2000: Williams and Wilkins PA., USA.

Concentrations and dosages will be dependent upon the condition being treated, the individual suffering from the condition, its severity, type, and duration, and the judgment of the attending health professional, e.g., physician. For use in enhanced wound healing, an “effective amount” of a therapeutic agent within the meaning of the present invention can be determined, e.g., by the individual, or by the individual's attending physician or veterinarian. Such amounts are readily ascertained by one of ordinary skill in the art and will enable enhanced wound healing when administered in accordance with the present invention. Factors which influence what a therapeutically effective amount will be include, the specific activity of the therapeutic agent being used, the wound type (mechanical or thermal, full or partial thickness, etc.), the size of the wound, the wound's depth (if full thickness), the absence or presence of infection, time elapsed since the injury's infliction, whether or not other wound treatment agents are used in combination with the TGF-β mimic, and the age, physical condition, existence of other disease states, and nutritional status of the patient. Additionally, other medication the patient may be receiving will effect the determination of the therapeutically effective amount of the therapeutic agent to administer.

The TGF-β mimic may be administered in any appropriate manner. In some embodiments, the TGF-β mimic is administered as a coating or integral component of an appliance used to close or otherwise treat a wound, such as a suture, staple, strap, and the like. Appliances used in treatment of wounds are discussed more fully herein, below. In some embodiments, the TGF-β mimic is applied in a matrix, as described for soft tissue augmentation. These embodiments are particularly useful in deep wounds where a set of sutures in deep tissue may be required, and a TGF-β mimic may be administered as part of the sutures, as a matrix, or by other conventional topical means, before the wound is closed. In some embodiments, such as for surgical wounds or very deep traumatic wounds, even deeper tissues or organs are breached, and the TGF-β mimic may be applied as part of the sutures, as a matrix, or by other conventional topical means, before the wound is closed.

In some embodiments, the TGF-β mimic is administered by injection, e.g., subcutaneous or intramuscular injection. In some embodiments, the TGF-β mimic may be administered by other enteral or parenteral routes. Concentrations of TGF-β mimic for non-topical administration can be, e.g., less than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.01, 0.1, 1, 5, or 10% (all concentration percentages given herein are w/w unless otherwise indicated). Further concentrations are given in the discussion of compositions, below.

In some embodiments, TGF-β mimic is administered topically. The TGF-β mimic may be administered neat (e.g., without excipients, e.g., as lyophilized powder). In these embodiments, typically the TGF-β mimic will be covered with an occlusive bandage, and will be suspended or dissolved in the perspiration trapped under the bandage.

More typically, the TGF-β mimic is administered in a lotion, gel, cream, or any other acceptable topical carrier, as apparent to the skilled artisan. Topical administration may be by any means that brings the TGF-β mimic and, optionally, other treatment agents, in contact with the surface of the skin, including application as a gel, lotion, cream, liposomal preparation or the like, with or without occlusion, or application as a plaster, patch, or similar device for extended contact with an affected area of skin. In embodiments of methods of the invention, the concentration of TGF-β mimic used for topical treatment may be more than about 0.00001, 0.00005, 0.0001, 0.001, 0.01, 0.1, 1, or 5%. In some embodiments, the concentration of the TGF-β mimic is more than about 0.00005%. The concentration of TGF-β mimic may be less than about 0.0001, 0.001, 0.001, 0.01, 0.1, 1, 5, or 10% (all concentration percentages given herein are w/w unless otherwise indicated). In some embodiments, the TGF-β mimic is administered topically at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

In topical administration, skin coverage may also be described in terms of total ng of TGF-β mimic/cm2 of skin; in these terms, a typical coverage per administration would be more than about 3, 6, 60, 600, 6000, 60,000, or 600,000 ng/cm2 of skin; less than about 900,000, 600,000, 60,000, 6000, 600, 60, or 6 ng/cm2 of skin; or about 6 ng/cm2 to about 600 ng/cm2 of skin; or about 60 ng/cm2 of skin.

For treatment of skin conditions or to produce a desired therapeutic effect, the frequency of administration of a formulation comprising a TGF-β mimic is dependent on factors including the nature of the formulation (e.g., concentration, presence or lack of other treatment agents, vehicle type), the severity and extent of the condition, and in some cases the judgment of a health care professional, e.g., attending physician.

Topical application may be more than about once, twice, three times, four times, five times, or six times per week, or more than once, twice, three times, four times, five times, or six times per day. Frequency of application may be less than about twice, three times, four times, five times, or six times per week, or less than about once, twice, three times, four times, five times, or six times per day. Some embodiments of the invention provide a method for enhanced wound healing in an individual by topical administration of an effective amount of a TGF-β mimic where the TGF-β mimic is administered an average of about once per day; in some embodiments, the TGF-β mimic is administered an average of about once or twice per day; in some embodiments, the TGF-β mimic is administered an average of about once to three times per day; in some embodiments, the TGF-β mimic is administered an average of more than about three times per day. In one embodiment, the TGF-β mimic is administered an average of about twice per day, typically in the morning upon rising and in the evening before retiring.

Topical administration may be without a covering. More typically, topical administration may include the use of a covering over the TGF-β mimic, which may be occlusive or non-occlusive.

The duration of treatment generally will depend on the response of the skin to therapeutic treatment. Treatment may continue at the discretion of the individual being treated or at the discretion of the attending health care professional, e.g., physician. In some cases, administration or application of a formulation containing a TGF-β mimic may be more frequent at the beginning of treatment and less frequent as treatment continues and the wound heals or the desired effect is achieved. In some cases, treatment may continue indefinitely in order to maintain a wound in abeyance or in an improved state, or to slow the progression of a wound. Indefinite treatment can be given in the case of, e.g., chronic wounds. These modifications of frequency and duration are easily accomplished by the individual being treated or by the attending health care professional, e.g., physician.

In some embodiments, the TGF-β mimic is administered as part of a wound dressing, either integrally associated with the dressing or as a coating on the dressing. Wound dressings are well-known in the art. These include, without limitation, films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer). See, e.g., Kannon and Garrett, Dermatol. Surg. 21:583-590 (1995); Davies, Burns 10:94 (1983).

In some embodiments, the methods of the invention provide a method of enhanced wound healing by administering to the wounded individual an effective amount of a TGF-β mimic. The TGF-β mimic may be administered by any appropriate means; in some embodiments, the TGF-β mimic is administered topically. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). In some embodiments, the TGF-β mimic is in a lotion comprising a mixture of emulsifying lanolin alcohols, waxes, and oils (e.g., EUCERIN™) or in a lotion comprising a mixture of petrolatum or mineral oil, a quaternary ammonium compound, a fatty alcohol, and a fatty ester emollient (e.g., CUREL™), as described below for compositions. The concentration of the TGF-β mimic (e.g., cytomodulin) can be more than about 0.00005%, 0.0001%; 0.001%; 0.01%; 0.1%; 1%, or 10%; the TGF-β mimic can be at a concentration of less than about 15%, 10%, 1%, 0.1%, 0.01%; 0.001%; or 0.0001%; in some embodiments, about 0.0001% to about 0.01%, in some embodiments about 0.001%. The lotion containing the TGF-β mimic (e.g., cytomodulin) is applied at a frequency of once to three times per day, in some embodiments once per day, until the desired result, e.g., healing of the wound, is observed. In some embodiments initial daily application may be followed by topical application once to three times per week, in some embodiments once per week, thereafter, until healing is complete.

In some embodiments, the methods of the invention provide a method of enhanced wound healing in an individual with a wound that results in reduced scarring of the wound site by administering to the wounded individual an effective amount of a TGF-β mimic. In some embodiments, the methods of the invention provide a method of enhanced wound healing in an individual with a wound that results in substantially no visible scarring of the wound site, i.e., scarless wound healing, by administering to the wounded individual an effective amount of a TGF-β mimic. The TGF-β mimic may be administered by any appropriate means; in some embodiments, the TGF-β mimic is administered topically. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). In some embodiments, the TGF-β mimic is in a lotion comprising a mixture of emulsifying lanolin alcohols, waxes, and oils (e.g., EUCERIN™) or a lotion comprising a mixture of petrolatum or mineral oil, a quaternary ammonium compound, a fatty alcohol, and a fatty ester emollient (e.g., CUREL™), as deiscribed below for compositions. The concentration of the TGF-β mimic (e.g., cytomodulin) can be more than about 0.00005%, 0.0001%; 0.001%; 0.01%; 0.1%; 1%, or 10%; the TGF-β mimic can be at a concentration of less than about 15%, 10%, 1%, 0.1%, 0.01%; 0.001%; or 0.0001%; in some embodiments, about 0.0001% to about 0.01%, in some embodiments about 0.001%. The lotion containing the TGF-β mimic (e.g., cytomodulin) is applied at a frequency of once to three times per day, in some embodiments once per day, until the desired result, e.g., healing of the wound with reduced scarring or scarless healing, is observed. In some embodiments initial daily application may be followed by topical application once to three times per week, in some embodiments once per week, thereafter, until healing is complete.

In some embodiments, the methods of the invention provide a method of enhanced healing of a burn wound, by administering to the wounded individual an effective amount of a TGF-β mimic. In some embodiments, the methods of the invention provide a method of enhanced healing of a burn wound that results in reduced or substantially no visible scarring of the wound site, by administering to the wounded individual an effective amount of a TGF-β mimic. The TGF-β mimic may be administered by any appropriate means; in some embodiments, the TGF-β mimic is administered topically. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). The methods may utilize the TGF-β mimic neat or in any pharmaceutically acceptable carrier appropriate to topical administration for burn wounds. The concentration of the TGF-β mimic (e.g., cytomodulin) can be more than about 0.00005%, 0.0001%; 0.001%; 0.01%; 0.1%; 1%, or 10%; the TGF-β mimic can be at a concentration of less than about 15%, 10%, 1%, 0.1%, 0.01%; 0.001%; or 0.0001%; in some embodiments, about 0.0001% to about 0.01%, in some embodiments about 0.001%. The TGF-β mimic (e.g., cytomodulin) is applied at a frequency of once to three times per day, in some embodiments once per day, until the desired result, e.g., healing of the burn wound, in some embodiments, with reduced scarring or scarless healing, is observed. In some embodiments initial daily application may be followed by topical application once to five times per week, in some embodiments once per week, or twice per week, or three times per week thereafter, until healing is complete.

In some embodiments, the methods of the invention provide a method of enhanced healing of a chronic wound, by administering to the wounded individual an effective amount of a TGF-β mimic. In some embodiments, the methods of the invention provide a method of enhanced healing of a chronic wound in a diabetic individual, by administering to the individual an effective amount of a TGF-β mimic. The TGF-β mimic may be administered by any appropriate means; in some embodiments, the TGF-β mimic is administered topically. In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1. In some embodiments, the methods utilize cytomodulin (SEQ ID NO: 1). The methods may utilize the TGF-β mimic neat or in any pharmaceutically acceptable carrier appropriate to topical administration for chronic wounds. The concentration of the TGF-β mimic (e.g., cytomodulin) can be more than about 0.00005%, 0.0001%; 0.001%; 0.01%; 0.1%; 1%, or 10%; the TGF-β mimic can be at a concentration of less than about 15%, 10%, 1%, 0.1%, 0.01%; 0.001%; or 0.0001%; in some embodiments, about 0.0001% to about 0.01%, in some embodiments about 0.001%. The TGF-β mimic (e.g., cytomodulin) is applied at a frequency of once to three times per day, in some embodiments once per day, until the desired result, e.g., healing of the chronic wound, in some embodiments, is observed. In some embodiments where the enhanced wound healing involves halting the spread of a chronic wound, e.g., where the wound does not heal completely or heals completely but very slowly, the application of TGF-β mimic may continue indefinitely. In some embodiments initial daily application may be followed by topical application once to five times per week, in some embodiments once per week, or twice per week, or three times per week thereafter, until healing is complete.

In some embodiments, the TGF-β mimic is used in combination with another wound care method (e.g., for debridement of burns, or the like) or agent. If the TGF-β mimic is used in combination with another wound care method or composition, any combination of the TGF-β mimic and the additional method or composition may be used. Thus, for example, if use of a TGF-β mimic is in combination with another wound care agent, the two may be administered simultaneously, consecutively, in overlapping durations, in similar, the same, or different frequencies, etc. In some cases a composition will be used that contains a TGF-β mimic in combination with one or more other wound care agents.

Other wound care agents that may be used in methods of the invention include, without limitation, animicrobials, antivirals, antiseptics, anesthetics, analgesics, antixidants, antiinflammatories, and antipruritics, which are described in more detail below. Dosages, routes of administration, administration regimes, and the like for these agents are well-known in the art.

III. Compositions

Another aspect of the present invention relates to compositions comprising a TGF-β mimic, optionally in combination with other desired ingredients. Such compositions can be used treat skin conditions, augment soft tissue, or to enhance wound healing (e.g., accelerate wound healing and/or promote healing of wounds with reduced or substantially no scarring). “Composition” and “formulation” are used interchangeably herein.

A. Composition Components

When a TGF-β mimic is prepared in a composition for administration by mixing with physiologically acceptable carriers, i.e., carriers which are non-toxic to recipients at the dosages and concentrations employed, this will normally entail combining the TGF-β mimic(s) with a pharmaceutically acceptable vehicle. Though in some embodiments, topical preparations need not be sterile, generally compositions of the invention, including topical compositions (especially for wound healing) must be sterile. This is readily accomplished by filtration through sterile filtration (0.22 micron) membranes, or by other art-accepted means.

1. TGF-β Mimics

Any suitable TGF-β mimic may be used in accordance with the description herein. A single TGF-β mimic may be used, or more than one TGF-β mimic may be used. In some embodiments, the TGF-β mimic is a peptide. In some embodiments, the TGF-β mimic used is that of SEQ ID NO: 1. In some embodiments, the TGF-β mimic used is that of SEQ ID NO: 2. In some embodiments, the TGF-β mimic used is that of SEQ ID NO: 3. In some embodiments, the TGF-β mimic used is that of SEQ ID NO: 11. In some embodiments, the TGF-β mimic used is that of SEQ ID NO: 13. In some embodiments, the TGF-β mimic used is that of SEQ ID NO: 14. In some embodiments, a TGF-β mimic of any of SEQ ID. NOS: 4-10, 12, or 15-42 may be used. In some embodiments, a combination of TGF-β mimics is used.

In some embodiments, the TGF-β mimic contains a peptide sequence that is at least about 70%, 80%, 90%, or 95% identical to one of SEQ ID NOS: 1-42, or SEQ ID NOS: 1, 2, 3, 1, 13, or 14, or SEQ ID NO: 1.

In compositions of the invention, the TGF-β mimic may be a free acid or free base, or may also be formulated as a pharmaceutically acceptable salt or pharmaceutically acceptable ester or amide. As used herein, the term “TGF-β mimic” includes all pharmaceutically acceptable salts or pharmaceutically acceptable esters or amides thereof. The term “pharmaceutically acceptable salt” or “pharmaceutically acceptable ester or amide” means those salts, esters, or amides that retain the biological effectiveness and properties of the compounds used in the present invention. For example, a pharmaceutically acceptable salt does not substantially interfere with the beneficial effect of a TGF-β mimic in, e.g., stimulating collagen synthesis.

TGF-β mimics form pharmaceutically acceptable salts with organic and inorganic acids and can be administered in salt form or the TGF-β mimic can be amidated. Examples of suitable acids for the formation of pharmaceutically acceptable salts are hydrochloric, sulfuric, phosphoric, acetic, benzoic, citric, malonic, salicylic, malic, fumaric, succinic, tartaric, lactic, gluconic, ascorbic, maleic, benzene-sulfonic, methane- and ethanesulfonic, hydroxymethane- and hydroxyethane-sulfonic.

Salts may also be formed with suitable organic pharmaceutically acceptable base addition salts. These organic bases form a class whose limits are readily understood by those skilled in the art. Merely for purposes of illustration, the class may be said to include mono-, di-, and trialkylamines, such as methylamine, dimethylamine, and triethylamine; mono-, di-, or trihydroxyalkylamines such as mono-, di-, and triethanolamine; amino acids such as arginine, and lysine; guanidine; N-methyl-glucosamine; N-methyl-glucamine; L-glutamine; N-methylpiperazine; morpholine; ethylenediamine; N-benzylphenethylamine; tris(hydroxymethyl)aminomethane; and the like. (See, for example, “Pharmaceutical Salts,” J. Pharm. Sci., 66(1), 1-19 (1977).)

2. Other Components

Other components besides a TGF-β mimic may be included in compositions of the invention, depending on its intended use.

Exemplary additional ingredients for treatment of dermatitis include steroid amcinonide, diflorasone diacetate, and hydrocortisone, antipruritics, and antibiotics.

Anti-acne ingredients include benzoyl peroxide, antibiotics, e.g., erythromycin, clindamycin phosphate, 5,7-dichloro-8-hydroxyquinoline, resorcinol, resorcinol acetate, salicylic acid, azaleic acid, long chain dicarboxylic acids, sulfur, zinc, retinoids, antiandrogens, and various natural agents such as those derived from green tree, and mixtures thereof. Other non-limiting examples of suitable anti-acne agents for use herein are described in U.S. Pat. No. 5,607,980, which description is incorporated herein by reference.

Ingredients useful in compositions for the treatment of urticaria include H1 antihistamine agents and H2 antihistamines, glucocorticosteroids, doxepin (e.g., at 5% in topical preparations), capsaicin, and cyproheptadine.

Exemplary ingredients for the treatment of psoriasis include agents such as shale oil and derivatives thereof, elubiol, ketoconazole, coal tar and petroleum distillates, salicylic acid, zinc pyrithione, selenium sulfide, hydrocortisone, sulfur, menthol, psoralen, pramoxine hydrochloride anthralin, and methoxsalen; calcipotriene, or tazarotene; steroids, such as 2-(acetyloxy)-9-fluoro-1′,2′,3′,4′-tetrahydro-11b-hydroxypregna-1,4-dieno[16,17-b]naphthalene-3,20-dione and 21-chloro-9-fluoro-1′,2′,3′,4′-tetrahydro-11-hydroxypregna-1,4-dieno[16z, 17-b]naphthalene-3,20-dione, and others including those that are antiinflammatories.

Exemplary ingredients for the treatment of rosacea include topical antibiotic, such as metronidazole, and oral antibiotics such as tetracycline, minocycline, erythromycin, and doxycycline.

For the treatment of mucositis, compositions may include topical pain relief agents, and oral pain relief agents such as paracetamol or stronger painkillers either by mouth or injection. Additional ingredients include allopurinol and vitamin E.

Additional ingredients useful in compositions for treatment of skin conditions and for enhanced wound healing include antiinflammatory agents, antiviral agents, antimicrobial agents, anesthetics and analgesics, antipruritics, and vitamins and antioxidants.

Antiinflammatory agents Anti-inflammatory agents of use in the invention include steroidal, non-steroidal, and other compounds.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone; dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Nonsteroidal anti-inflammatory agents are also suitable for use herein as skin agents in the compositions of the invention. Non-limiting examples of non-steroidal anti-inflammatory agents suitable for use herein include oxicams (e.g., piroxicam, isoxicam, tenoxicam, sudoxicam, CP-14,304); salicylates (e.g., aspirin, disalcid, benorylate, trilisate, safapryn, solprin, diflunisal, fendosal); acetic acid derivatives (e.g., diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac); fenamates (e.g., mefenamic, meclofenamic, flufenamic, nifluric, tolfenamic acids); propionic acid derivatives (e,g., ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofenic); pyrazoles (e.g., phenylbutazone, oxyphenbutazone, feprazone, azapropazone, trimethazone); and combinations thereof as well as any dermatologically acceptable salts or esters of thereof. COX-2 inhibitors are also suitable for use herein, and include, but are not limited to, AZD 3582 (ASTRAZENECA and NicOx), Celecoxib (PHARMACIA Corp.) (4-[5-(4-methylphenyl)-3-(trifluoromethyl)-1H-pyrazol-1-yl] benzenesulfonamide), Meloxicam (BOEHRINGER INGELHEIM Pharmaceuticals) (4-hydroxy-2-methyl-N-(5-methyl-2-thiazolyl)-2H-1,2GW-406381 (GLAXOSMITHKLINE), Etoricoxib (MERCK & Co.), Rofecoxib (MERCK & Co.) (4-[4-(methylsulfonyl) phenyl]-3-phenyl-2(5H)-furanone), Lumiracoxib (NOVARTIS Pharma AG), Valdecoxib (PHARMACIA Corp.) (4-(5-methyl-3-phenyl-4-isox-azolyl) benzenesulfonamide), and Etodolac (WYETH Ayerst Laboratories) ((.+-.) 1,8-diethyl-1,3,4,9-tetrahydropyrano-[3,4-b]acid).

Other non-limiting examples of suitable anti-inflammatory or similar other skin agents include candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commniphora, particularly Commniphora Mukul), kola extract, chamomile, red clover extract, sea whip extract, anise oil, garlic oil, ginger extract, vasoconstrictors such as phenylephrine hydrochloride, and combinations thereof.

Further non-limiting examples of suitable anti-inflammatory or similar other skin agents include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific non-limiting examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, and combinations thereof.

Antiviral agents Compositions and methods of the invention may include antiviral agents. Suitable anti-viral agents include, but are not limited to, metal salts (e.g., silver nitrate, copper sulfate, iron chloride, etc.) and organic acids (e.g., malic acid, salicylic acid, succinic acid, benzoic acid, etc.).

Antimicrobials Anti-microbial agents useful in compositions and methods of the invention include antifungal, antibacterial, and antiseptic compounds.

Antifungal compounds include, but are not limited to, compounds such as imidazole antifungals. Specific antifungals include butocouazole nitrate, miconazole, econazole, ketoconazole, oxiconizole, haloprogin, clotrimazole, and butenafine HCl, naffifine, terbinafine, ciclopirox, and tolnaftate.

Antibacterial and antiseptic compounds include phenol-TEA complex, mupirocin, triclosan, chlorocresol, chlorbutol, iodine, clindamycin, CAE (Anjinomoto Co., Inc., containing DL-pyrrolidone Carboxylic acid salt of L-Cocoyl Arginine Ethyl Ester), povidone-iodine, polymyxin b sulfate-bacitracin, zinc-neomycin sulfate-hydrocortisone, chloramphenicol, methylbenzethonium chloride, and erythromycin and antiseptics (e.g., benzalkonium chloride, benzethonium chloride, chlorhexidine gluconate, mafenide acetate, nitroftirazone, nitromersol and the like may be included in compositions of the invention. Antiparasitics, such as lindane may also be included.

Further examples of antimicrobial and antifuingal agents useful in the present invention include, but are not limited to, β-lactum drugs, quinolone drugs, ciprofloxacin, norfloxacin, tetracycline, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole.

In particular, topical antibiotics suitable for use in the invention chloramphenicol, chlortetracycline, clyndamycin, clioquinol, erythromycin, framycetin, gramicidin, fusidic acid, gentamicin, mafenide, mupiroicin, neomycin, polymyxin B, bacitracin, silver sulfadiazine, tetracycline and chlortetracycline.

In addition, in some embodiments it may be desirable to administer systemic antibiotics in combination with administration of a composition of the invention. Any suitable systemic antibiotic known in the art and suitable for use with a particular individual being treated may be used in combination with the compositions of the invention.

Anesthetics and Analgesics Anesthetic substances of use in the invention include butamben picrate, lidocaine, xylocaine, benzocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Analgesic agents include dyclonine hydrochloride, aloe vera, fentanyl, capsaicin, and the like.

Antipruritics Anti-pruritic agents include alclometasone dipropionate, betamethasone valerate, and isopropyl myristate MSD.

Vitamins and antioxidants Vitamins and antioxidants may also be used in combination with the compositions and methods of the invention. These include vitamins K, E, and C. Addition of vitamin K may promote wound healing, and the antioxidant vitamins C and E also have beneficial effects on wound healing and may reduce scar formation. See, e.g., U.S. Pat. No. 6,187,743.

The compositions and methods of the present invention may utilize a wide range of additional components. The CTFA Cosmetic Ingredient Handbook, Seventh Edition, 1997 and the Eighth Edition, 2000, which are incorporated by reference herein in its entirety, describes a wide variety of ingredients commonly used in skin care compositions and methods, which are suitable for use in the compositions of the present invention. Other topically-useful compounds are listed in Remington's Pharmaceutical Sciences, 20th Ed., Lippincott Williams & Witkins, Baltimore, Md. (2000) (hereinafter Remington's), U.S. Pharmacopeia and National Formulary, The United States Pharmacopeial Convention, Inc., Rockville, Md. and Physician's Desk Reference, Medical Economics Co., Inc., Oradell, N.J. incorporated herein by reference. The concentration of the other ingredient in formulations provided by the invention is that which provides an effective amount of the other ingredient; these concentrations are well-known in the art. See, e.g., the above references, as well as Textbook of Dermatology, Champion, Burton, Burns, and Bretnach, eds., Blackwell Publishing, 1998.

B. Compositions for Treatment of Skin Conditions and Enhanced Wound Healing

In some embodiments, the invention provides compositions for the treatment of skin conditions and/or for enhanced wound healing that are topical compositions. The topical compositions may be non-oral topical compositions or oral topical compositions. The latter are suitable for treatment of oral wounds or skin conditions that include the mouth, such as mucositis. In other embodiments, the invention provides compositions for the treatment of skin conditions and/or for enhanced wound healing that are suitable for injection or other non-topical uses. These latter compositions utilize pharmaceutically acceptable carriers that are well-known in the art. See, e.g., Remington's Pharmaceutical Sciences, Gennaro, A R, ed., 20th edition, 2000: Williams and Wilkins PA., USA. In addition, some wound healing compositions comprise a TGF-β mimic associated with a wound healing appliance (e.g., sutures, staples, and the like).

1. Topical Non-Oral Compositions

In methods for treatment of skin conditions, a TGF-β mimic is preferably applied in a formulation suitable for topical use. Numerous vehicles for topical application of active compositions are known in the art. See, e.g., Remington's Pharmaceutical Sciences, Gennaro, A R, ed., 20th edition, 2000: Williams and Wilkins PA., USA. All suitable compositions usually employed for topically administering therapeutics may be used, e.g., creams, lotions, gels, dressings, shampoos, tinctures, pastes, ointments, salves, powders, liquid or semiliquid formulation, patches, liposomal preparations, and the like. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used. The TGF-β mimic may optionally be dissolved in a small amount (e.g., less than 100, less than 10, or less than 1 ul/mg TGF-β mimic; in some embodiments, less than 1 ul/mg TGF-β mimic) of an appropriate solvent, such as ethanol or DMSO, before dispersion in the vehicle; however, this is not required.

Compositions known in the art, preferably hypo allergic and pH controlled are especially preferred for topical administration, and include toilet waters, packs, lotions, skin milks or milky lotions. The preparations contain, besides the TGF-β mimic and, optionally, other ingredients, components usually employed in such preparations. Examples of such components are oils, fats, waxes, surfactants, humectants, thickening agents, antioxidants, viscosity stabilizers, chelating agents, buffers, preservatives, perfumes, dyestuffs, lower alkanes, and the like.

Examples of oils comprise fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearate, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

In some embodiments, the invention provides a composition containing a TGF-β mimic in a gel. Gels comprise a water-insoluble material, where the water-insoluble material forms a gel with the water of the formulation. The material is therefore hydrophilic but does not dissolve in water to any great extent. The material can be a polymeric material, for example, a water-absorbing non water-soluble polymer.

Gels typically comprise a base selected from an oleaginous base, water, or an emulsion-suspension base. To the base is added a gelling agent which forms a matrix in the base, increasing its viscosity. Examples of gelling agents are polymers such as hydroxypropyl cellulose, acrylic acid polymers, and the like. However, non-polymeric materials that form gels with water can also be used, e.g. clays such as kaolin or bentonite. Customarily, the active ingredient (compounds) is added to the formulation at the desired concentration at a point preceding addition of the gelling agent. The amount of compound incorporated into a topical formulation is not critical; the concentration should be within a range sufficient to permit ready application of the formulation to the affected tissue area in an amount that will deliver the desired amount of compound to the desired treatment site. The customary amount of a topical formulation to be applied to an affected tissue will depend upon an affected tissue size and concentration of compound in the formulation.

In some embodiments, a TGF-β mimic may also be encapsulated in the biodegradable polymer. Biodegradable polymers are usually based on functional groups such as esters, anhydrides, orthoesters, and amides. Rapidly biodegradable polymers include poly[lactide-co-glycolide], polyanhydrides, and polyorthoesters. Preferred bioerodible polymers include polylactides, polyglycolides, and copolymers thereof, poly(ethylene terephthalate), poly(butyric acid), poly(valeric acid), poly(lactide-co-caprolactone), poly(lactide-co-glycolide), polyanhydrides, polyphosphazenes, poly(c-caprolactone), poly(dioxanone), poly(hydroxybutyrate), poly(hydroxyvalerate), polyorthoesters, blends, and copolymers thereof. Examples of biodegradable and biocompatible polymers of acrylic and methacrylic acids or esters include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), etc. Other polymers which can be used in the present invention include polyalkylenes such as polyethylene and polypropylene; polyarylalkylenes such as polystyrene; poly(alkylene glycols) such as poly(ethylene glycol); poly(alkylene oxides) such as poly(ethylene oxide); and poly(alkylene terephthalates) such as poly(ethylene terephthalate). Additionally, polyvinyl polymers can be used which include polyvinyl alcohols, polyvinyl ethers, polyvinyl esters, and polyvinyl halides. Exemplary polyvinyl polymers include poly(vinyl acetate), polyvinyl phenol, and polyvinylpyrrolidone. Mixtures of two or more of the above polymers could also be used in the present invention

Some polymeric materials are known to release entrapped compounds upon exposure to a stimulus such as a change in pH or temperature. An examples of microparticles that release as a function of a change in pH include the diketopiperazine particles described in U.S. Pat. No. 5,352,461, and the proteinoid formulations described in U.S. Pat. Reissue No. 35,862.

The non-oral topical compositions may also contain conventional additives employed in those products. Conventional additives include humectants, emollients, lubricants, stabilizers, dyes and other coloring agents, and perfumes, providing the additives do not interfere with the therapeutic properties of the composition.

In accordance with this invention, therapeutically effective amounts of the compositions of the present invention may be admixed with a non-oral topical vehicle to form a topical composition. These amounts are readily determined by those skilled in the art without the need for undue experimentation.

For preparing compositions for topical administration, the concentration of TGF-β mimic may be more than about 0.00001, 0.00005, 0.0001, 0.001, 0.01, 0.1, 1, or 5%; in some embodiments, the concentration of the TGF-β mimic is more than about 0.00005%; the concentration of TGF-β mimic may be less than about 0.0001, 0.001, 0.001, 0.01, 0.1, 1, 5, or 10% (all concentration percentages given herein are w/w unless otherwise indicated). In some embodiments, the TGF-β mimic is incorporated at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

In some embodiments, one or more TGF-β mimics, optionally with other ingredients, may be in a lotion or cream containing a mixture of emulsifying lanolin alcohols, waxes, and oils. One such commercially available lotion is EUCERIN™ Dry Skin Therapy Original Moisturizing Lotion, which contains water, mineral oil, isopropyl myristate, PEG40 sorbitan peroleate, glyceryl lanolate, sorbitol, propylene glycol, cetyl palmitate, magnesium sulfate, aluminum stearate, lanolin alcohol, BHT, methylchloroisothiazolinone, and methylisothiazolinone. In some embodiments, one or more TGF-β mimics, optionally with other ingredients, may be in a lotion or cream containing a mixture of petrolatum or mineral oil, a quaternary ammonium compound, fatty alcohol, and fatty ester emollient. One such commercially available lotion is CUREL™ Fragrance Free Daily Moisturizing Lotion, which contains water, glycerin, distearyldimonium chloride, petrolatum, isopropyl palmitate, cetyl alcohol, dimethicone, sodium chloride, methylparaben, and propylparaben.

Some embodiments of compositions of the invention comprise a TGF-β mimic (e.g., cytomodulin (SEQ ID NO: 1)) in a lotion or cream containing a mixture of emulsifying lanolin alcohols, waxes, and oils (e.g., EUCERIN™ Dry Skin Therapy Original Moisturizing Lotion) at a concentration greater than about 0.00005%. Some embodiments of compositions of the invention comprise a TGF-β mimic (e.g., cytomodulin (SEQ ID NO: 1)) in a lotion or cream containing a mixture of emulsifying lanolin alcohols, waxes, and oils (e.g., EUCERIN™ Dry Skin Therapy Original Moisturizing Lotion) at a concentration of about 0.0001% to about 0.01%. Some embodiments comprise a TGF-β mimic (e.g., cytomodulin (SEQ ID NO: 1)) in a lotion or cream containing a mixture of emulsifying lanolin alcohols, waxes, and oils (e.g., EUCERIN™ Dry Skin Therapy Original Moisturizing Lotion) at a concentration of about 0.001%. Some embodiments of compositions of the invention comprise a TGF-β mimic (e.g., cytomodulin (SEQ ID NO: 1)) in a lotion or cream containing a mixture of petrolatum or mineral oil, a quaternary ammonium compound, a fatty alcohol, and a fatty ester emollient (e.g., CUREL™ Fragrance Free Daily Moisturizing Lotion) at a concentration greater than about 0.00005%. Some embodiments of compositions of the invention-comprise a TGF-β mimic (e.g., cytomodulin (SEQ ID NO: 1)) in a lotion or cream containing a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.0001% to about 0.01%. Some embodiments comprise a TGF-β mimic (e.g., cytomodulin (SEQ ID NO: 1)) in a lotion or cream containing a mixture of petrolatum or mineral oil, a quaternary ammonium compound, a fatty alcohol, and a fatty ester emollient (e.g., CUREL™ Fragrance Free Daily Moisturizing Lotion) at a concentration of about 0.001%.

In addition, a TGF-β mimic and, optionally, other ingredients, may be formulated in liposome-containing compositions. Liposomes are artificial vesicles formed by amphipathic molecules such as polar lipids, for example, phosphatidyl cholines, ethanolamines and serines, sphingomyelins, cardiolipins, plasmalogens, phosphatidic acids and cerebrosides. Liposomes are formed when suitable amphipathic molecules are allowed to swell in water or aqueous solutions to form liquid crystals usually of multilayer structure comprised of many bilayers separated from each other by aqueous material (also referred to as coarse liposomes). Another type of liposome known to be consisting of a single bilayer encapsulating aqueous material is referred to as a unicellular vesicle. If water-soluble materials are included in the aqueous phase during the swelling of the lipids they become entrapped in the aqueous layer between the lipid bilayers.

The incorporation of TGF-β mimics into liposomal preparations can be achieved by a number of methods. With respect to liposomal preparations, any known methods for preparing liposomes for treatment of a condition may be used. See, for example, Bangham et al., J. Mol. Biol, 23: 238-252 (1965) and Szoka et al., Proc. Natl Acad. Sci. 75: 4194-4198 (1978). Ligands may also be attached to the liposomes to direct these compositions to particular sites of action.

Liposomes containing a TGF-β mimic and, optionally, other ingredients can be employed directly or they can be employed in a suitable pharmaceutically acceptable carrier, e.g., for topical administration. The viscosity of the liposomes can be increased by the addition of one or more suitable thickening agents such as, for example xanthan gum, hydroxypropyl cellulose, hydroxypropyl methyl cellulose and mixtures thereof. The aqueous component may consist of water alone or it may contain electrolytes, buffered systems and other ingredients, such as, for example, preservatives. Suitable electrolytes which can be employed include metal salts such as alkali metal and alkaline earth metal salts. Exemplary metal salts are calcium chloride, sodium chloride and potassium chloride. The concentration of the electrolyte may vary from zero to 260 mM, preferably from 5 mM to 160 mM. The aqueous component is placed in a suitable vessel which can be adapted to effect homogenization by effecting great turbulence during the injection of the organic component. Homogenization of the two components can be accomplished within the vessel, or, alternatively, the aqueous and organic components may be injected separately into a mixing means which is located outside the vessel. In the latter case, the liposomes are formed in the mixing means and then transferred to another vessel for collection purpose.

The organic component consists of a suitable non-toxic, pharmaceutically acceptable solvent such as, for example ethanol, glycerol, propylene glycol and polyethylene glycol, and a suitable phospholipid which is soluble in the solvent. Suitable phospholipids which can be employed include lecithin, phosphatidylcholine, phosphatidylserine, phosphatidylethanolamine, phosphatidylinositol, lysophosphatidylcholine and phosphatidyl glycerol, for example. Other lipophilic additives may be employed in order to selectively modify the characteristics of the liposomes. Examples of such other additives include stearylamine, phosphatidic acid, tocopherol, cholesterol and lanolin extracts. In addition, other ingredients which can prevent oxidation of the phospholipids may be added to the organic component. Examples of such other ingredients include tocopherol, butylated hydroxyanisole, butylated hydroxytoluene, ascorbyl palmitate and ascorbyl oleate. Preservatives such a benzoic acid, methyl paraben and propyl paraben may also be added.

Although TGF-β mimics are generally capable of penetrating cell membranes and reaching the deep layers of skin, it may be useful in some embodiments to also include a penetration enhancer in the formulations of the invention. A penetration enhancer is a substance that improves cutaneous penetration of a bioactive substance. Suitable penetration enhancers include, for example, dimethyl sulfoxide (DMSO), DMSO-like compounds, ethanolic compounds, pyroglutamic acid esters and other solvents or compounds known to those skilled in the pharmaceutical art which facilitate dermial penetration of the drugs or chemicals chosen for the pharmaceutical composition. Other penetration enhancers include amphiphiles such as L-amino acids, anionic surfactants, cationic surfactants, amphoteric surfactants, nonionic surfactants, fatty acids and alcohols. Additional penetration enhancers are disclosed in Remington: The Science and Practice of Pharmacy, 19th Edition (1995) on page 1583. The penetration enhancer chosen and the relative proportion of the penetration enhancer with respect to the active drugs or chemicals depends on the desired rate of delivery of the drugs or chemicals into the skin, which in turn depends on the condition being treated and the outcome sought. More specifically, the type and amount of enhancer is chosen so that a sufficiently high concentration of active drugs or chemicals is attained in the skin to treat the condition within the time period considered desirable.

2. Topical Oral Compositions

In another form of the invention, a TGF-β mimic is incorporated into an oral topical vehicle which may be in the form of, e.g. a mouthwash, rinse, oral spray, suspension, dental gel, and the like. Typical nontoxic oral vehicles known in the pharmaceutical arts may be used in the present invention. In some embodiments, the oral vehicles are water, ethanol, and water-ethanol mixtures. The water-ethanol mixtures are generally employed in a weight ratio from about 1:1 to about 20:1, preferably from about 3:1 to about 20:1, and most preferably from about 3:1 to about 10:1, respectively. The pH value of the oral vehicle is generality from about 4 to about 7, and preferably from about 5 to about 6.5. An oral topical vehicle having a pH value below about 4 is generally irritating to the oral cavity and an oral vehicle having a pH value greater than about 7 generally results in an unpleasant mouth feel.

The oral topical TGF-β mimic compositions may also contain conventional additives normally employed in those products. Conventional additives include fluorine providing compound, a sweetening agent, a flavoring agent, a coloring agent, a humectant, a buffer, and an emulsifier, providing the additives do not interfere with the therapeutic properties of the TGF-β mimic composition.

Suitable buffer solutions useful in the oral topical therapeutic wound healing compositions include citric acid-sodium citrate solution, phosphoric acid-sodium acetate solution in amounts up to about 1%, and preferably from about 0.05% to about 0.5% by weight of the oral topical therapeutic wound healing composition.

For preparing compositions for oral topical administration, the concentration of TGF-β mimic may be more than about 0.00001, 0.00005, 0.0001, 0.001, 0.01, 0.1, 1, or 5%; in some embodiments, the concentration of the TGF-β mimic is more than about 0.00005%; the concentration of TGF-β mimic may be less than about 0.0001, 0.001, 0.001, 0.01, 0.1, 1, 5, or 10% (all concentration percentages given herein are w/w unless otherwise indicated). In some embodiments, the TGF-β mimic is incorporated at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

3. Wound Healing Appliances

In one form of the invention, a TGF-β mimic is incorporated into, or associated with, a wound healing appliance which may be in the form of, e.g., sutures, staples, gauze, bandages, burn dressings, artificial skins, liposome or micelle formulations, microcapsules, aqueous vehicles for soaking gauze dressings, and the like, and mixtures thereof. A variety of traditional ingredients may optionally be included in the wound healing appliance, or in the associated or incorporated TGF-β mimic, in effective amounts such as buffers, preservatives, toxicity adjusting agents, antioxidants, polymers for adjusting viscosity or for use as extenders, and excipients, and the like. Specific illustrative examples of such traditional ingredients include acetate and borate buffers, thimerosal, sorbic acid, methyl and propyl paraben and colorobutanol preservatives; sodium chloride and sugars to adjust the toxicity; and excipients such as mannitol, lactose and sucrose. Other conventional pharmaceutical additives known to those having ordinary skill in the pharmaceutical arts may also be used in the pharmaceutical composition.

In some embodiments the invention provides one or more TGF-β mimics associated with a solid support. In some embodiments, the solid support is a wound suture or staple. In some embodiments, the solid support is an artificial skin. In some embodiments, the solid support is a skin covering or wound dressing. In some embodiments, the solid support is an absorbent material, e.g., attached to an adhesive strip. The TGF-β mimic may be associated covalently or non-covalently with the support. If associated covalently, the covalent attachment may be direct or by means of a linker. If associated non-covalently, the TGF-β mimic may be associated directly through non-covalent forces, or it may be dissolved or in a carrier that is associated through non-covalent forces. In some embodiments, the skin coverings or wound dressings of the invention can provide slow or timed release of the TGF-β mimic, into a wound. Skin coverings and dressing materials can be any suitable material used in the art including bandage, gauze, sterile wrapping, hydrogel, hydrocolloid and similar materials.

In accordance with this invention, therapeutically effective amounts of the TGF-β mimics of the present invention may be employed in the wound healing appliance. These amounts are readily determined by those skilled in the art without the need for undue experimentation. The exact amount of TGF-β mimic employed is subject to such factors as the type and concentration of the TGF-β mimic and the type of wound healing appliance employed. Thus, the amount of TGF-β mimic will be varied in order to obtain the result desired in the final product and such variations are within the capabilities of those skilled in the art without need for undue experimentation. When the TGF-β mimic is attached directly to an appliance, the density of attachment is determined to provide optimum or desired effects, e.g., rapid or nonscarring wound healing. When the TGF-β mimic is attached indirectly to an appliance, e.g., in a carrier, the concentration of the TGF-β mimic can be any suitable concentration described herein for topical compositions.

4. Compositions for Tissue Augmentation

For use in methods of augmentation, in some embodiments a TGF-β mimic may be delivered to the desired site, e.g. a sphincter or an area for which cosmetic enhancement is desired, in a pharmaceutically acceptable carrier. However, generally the TGF-β mimic is in conjunction with a matrix. As used herein, a “matrix” refers to an insoluble molecular or supramolecular framework to which a TGF-β mimic may be attached, either covalently or non-covalently. Preferably, the formulations include a matrix that is capable of providing a structure for developing soft tissue, including cellular and extracellular components. Potential matrices may be biodegradable (resorbable) or nonbiodegradable (non-resorbable), and may be chemically or biologically defined.

For one example, the matrix can be inert, solid and non-porous, such as known and presently used as vessels for cell culture. Another form that may be taken by matrices of this invention is that of soluble polymers. Other suitable matrices for practice of this invention include various polymers and hydrogels. Such composites are useful in constructing templates for augmentation of soft tissue.

Composites of the invention can thus be made with resorbable polymers of various kinds, having TGF-β mimic carried by or grafted onto the lattice of the polymeric material. Of course, polymeric supports that are limited in resorbable properties such as hydroxyethyl methacrylate, polymethylmethacrylate, and N-vinylpyrrolidone methylmethacrylate, as a few examples, are also feasible. The composites can then be implanted in the area desired to be augmented; e.g., for cosmetic augmentation, the breast, or for sphincter augmentation, the sphincter.

Among the known and suitable resorbable hydrogels are combinations of polylactate and polyglycollate. Compounds of the invention can be covalently bound to such materials during synthesis of the polymers themselves or the polymers can be hydrolyzed such that attachment sites are available by irradiating the polymer or by chemically activating the polymer to generate free radicals. Then conventional techniques for grafting, or immobilizing, peptides onto polymer supports can be utilized to prepare composites useful in the practice of the methods of the invention. Resorbable hydrogels or polymers so prepared are particularly usefull for soft tissue augmentation. Examples of implants comprising resorbable and non-resorbably matrices in conjunction with other bioactive materials that may be used in the methods of the invention and techniques for their use are found in, e.g., U.S. Pat. Nos. 6,214,045; 6,638,308.

In accordance with this invention, therapeutically effective amounts of the TGF-β mimics of the present invention may be employed in the tissue augmentation matrix. These amounts are readily determined by those skilled in the art without the need for undue experimentation. The exact amount of TGF-β mimic employed is subject to such factors as the type and concentration of the TGF-β mimic and the type of matrix-building material employed. Thus, the amount of TGF-β mimic will be varied in order to obtain the result desired in the final product and such variations are within the capabilities of those skilled in the art without need for undue experimentation.

5. Other Compositions

The compositions are also prepared as injectables, either as liquid solutions or suspensions, e.g., for subcutaneous or intramuscular injection. The TGF-β mimic may also be systemically administered, for example, intravenously or intraperitoneally by infusion or injection. Solutions of the TGF-β mimic(s) can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The pharmaceutical dosage forms suitable for injection or infusion or topical application can include sterile aqueous solutions or dispersions or sterile powders comprising the TGF-β mimic that are adapted for the extemporaneous preparation of sterile injectable or infusible solutions or dispersions, optionally encapsulated in liposomes.

In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof The proper fluidity can be maintained, for example, by the formation of liposomes, by the maintenance of the required particle size in the case of dispersions or by the use of surfactants. The prevention of the action of mucroorganisms can be brought about by various antibacterial and antifungal agents, for example, parabens, chlorobutanol, phenol, sorbic acid, thimerosal, and the like.

In some cases, one of skill in the art may choose to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the TGF-β mimic(s) in the required amount in the appropriate solvent with various of the other ingredients enumerated above, as required, followed by filter sterilization. In the case of sterile powders for the preparation of sterile injectable solutions, methods of preparation include vacuum drying and the freeze-drying techniques, which yield a powder of the active ingredient plus any additional desired ingredient present in the previously sterile-filtered solutions.

For preparing compositions for administration by injection, the concentration of TGF-β mimic may be more than about 0.00001, 0.00005, 0.0001, 0.001, 0.01, 0.1, 1, or 5%; in some embodiments, the concentration of the TGF-β mimic is more than about 0.00005%; the concentration of TGF-β mimic may be less than about 0.0001, 0.001, 0.001, 0.01, 0.1, 1, 5, or 10%. In some embodiments, the TGF-β mimic is incorporated at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

In some instances, the TGF-β mimic(s) can also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the TGF-β mimic (s) may be combined with one or more excipients and used in the form of ingestible tablets, buccal tablets, troches, capsules, elixirs, suspensions, syrups, wafers, and the like.

The amount of active compound in such therapeutically useful compositions is such that an effective dosage level will be obtained. For preparing compositions for oral administration, the concentration of TGF-β mimic may be more than about 0.00001, 0.00005, 0.0001, 0.001, 0.01, 0.1, 1, or 5%; in some embodiments, the concentration of the TGF-β mimic is more than about 0.00005%; the concentration of TGF-β mimic may be less than about 0.0001, 0.001, 0.001, 0.01, 0.1, 1, 5, or 10%. In some embodiments, the TGF-β mimic is incorporated at a concentration of about 0.00001% to about 1%; or about 0.00001% to about 0.1%; or about 0.0001% to about 0.01%; or about 0.0005% to about 0.005%; or about 0.0005% to about 0.002%; or about 0.001%. In some embodiments, lower and higher concentrations, e.g. 0.00001-0.001%, and 0.001-0.01% are contemplated. In some embodiments, even higher concentrations may be warranted, e.g. 0.01%-0.1% or 0.1%-1%, or 0.1%-5%, or 0.1%-10%.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the therapeutic agent may be incorporated into sustained-release preparations and devices.

Useful solid carriers include finely divided solids such as talc, clay, microcrystalline cellulose, silica, alumina and the like. Useful liquid carriers include water, alcohols or glycols or water, alcohol/glycol blends, in which the present compounds can be dissolved or dispersed at effective levels, optionally with the aid of non-toxic surfactants.

Adjuvants such as fragrances and additional antimicrobial agents can be added to optimize the properties for a given use.

The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

IV. Kits of the Invention

In still another aspect, the present invention provides kits for the treatment of skin conditions, for tissue augmentation, or for enhanced wound healing. These kits comprise a compound or compounds described herein in a container and, optionally, instructions teaching the use of the kit according to the various methods and approaches described herein. Such kits may also include information, such as scientific literature references, package insert materials, clinical trial results, and/or summaries of these and the like, which indicate or establish the activities and/or advantages of the composition. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### EXAMPLES

### Example 1

Inhibition of DNA Synthesis of Mv-1-Lu Mink Lung Epithelial Cells

The effect of TGF-β and cytomodulin were evaluated by determining the rate of [1H]thymidine incorporation into total acid-insoluble DNA and cell number. See generally, Sampath et al., J. Biol. Chem., 267, pp. 20352-20362 (1992). DNA synthesis rates were determined in triplicate cultures after 24 hour treatment with various concentrations (10-9 M to 10-6 M) of either TGF-β or cytomodulin (which was synthesized by the Merrifield method) by adding [methyl-3H]thymidine (2 uCi/ml, 80 Ci/mmol) for 6 hours before the termination of the culture. Incorporation was terminated by aspiration of the medium, and after washing three times with phosphate-buffered saline, the trichloroacetic acid (10%)-precipitated radioactive DNA was extracted with 1.0% (w/v) sodium dodecyl sulfate, 0.1 M NaOH and quantitated by liquid scintillation counting. For cell number determination, 1×105 cells were plated in flasks in MEM containing 10% FBS, and after 24 hours, the growth medium was replaced with serum-free medium containing various conceptions of TGF-β and cytomodulin. Triplicate cultures were harvested every 24 hours for the duration of 7 days, and the cell number was determined by counting cells released by trypsin digestion in a fixed volume hemacytometer.

The growth inhibition curve for cytomodulin were similar to that observed for TGF-β at the same concentration range.

### Example 2

Growth and Colony Formation by NRK-49 F Fibroblasts in Soft Agar

The original assay for TGF-β, the ability to promote anchorage independent growth of normal fibroblasts is still one of the hallmarks of TGF-β activity. NRK49 F fibroblasts were grown at 37° C. in DEM supplemented with 10% fetal calf serum. The experiments were performed with culture medium, 10 ng/mg epidermal growth factor (EGF), and 10 ng/ml platelet-derived growth factor (PDGF); however, unlike TGF-β, which does not induce colony formation in the absence of these factors (see, for example, Massagu, J. Biol. Chem., 259, pp. 9756-9761 (1984)), cytomodulin did induce colony formation without these two growth factors. To this, either 100 nM TGF-β (positive control) or 100 nM cytomodulin was added. NRK49 F fibroblasts (5×104 cells/ml) were mixed with 0.3% agar were plated on the bottom of 35 mm culture dishes. Colony formation was observed starting on day 3 of culture.

As expected no colonies were formed in those cultures containing only the basic medium. Also, as expected, colonies with TGF-β grew colonies. Surprisingly, the cytomodulin cultures also formed colonies to approximately the same extent as the TGF-β cultures. The growth characteristics of the colonies over time were similar between TGF-β and cytomodulin cultures.

### Example 3

Further TGF-β Mimics

FIGS. 1A and 1B show the atomic coordinates of the bioactive structure of cytomodulin (atoms 1-101). Thus, the structure represented by FIGS. 1A and 1B, describes one of the possible structures consistent with TGF-β activity

Using the three dimensional structure of cytomodulin (FIGS. 1A and 1B) as a guide, cytomodulin analogs were designed and tested for TGF-β activity. An exemplary general formula to produce a peptide with atomic coordinates substantially the same as that of FIGS. 1A and 1B, which was used in the construction of these two cytomodulin analogs, was AAi-AAi+1-AAi+2 . . . -AAi+n:

(1) a hydrophobic or neutral amino acid at position i;

(2) a branched hydrophobic at position i+1;

(3) a small aliphatic at position i+2, where positions i+1 and i+2 together form the U-bend (β-bend) structure; and

(4) a side-chain possessing a hydrogen bond acceptor shortly thereafter (at position AAi+n)

Initially, two cytomodulin analogs, L-I-A-E-A-K (SEQ ID NO: 2 or L2) and L-I-A-P-E-A (SEQ ID NO:3 or L1) were synthesized and tested. In both peptides, -V-A- was replaced by -I-A- and the first two N-terminal amino acid sequence of cytomodulin was replaced with leucine. In SEQ ID NO:2, glutamic acid is at position i+3 since it is the first side-chain after the U (β)-bend structure. In SEQ ID NO:3, proline is at position i+3. Since proline does not have a “side-chain,” the glutamic acid was placed at position i+4.

Both cytomodulin analogs, L1 and L2, displayed at least as much TGF-β like activity as cytomodulin. They promoted the growth of NRK-49F cells in soft agar and inhibited the proliferation of MV-1-Lu cells. They increased the expression of type I collagen and TGF-β and decreased the expression of collagenase in human dermal fibroblasts. Moreover, as with cytomodulin, L1 and L2 also increased the expression of type I collagen, TGF-β and alkaline phosphatase in HOS cells.

Further analogs of SEQ ID NO:2 and SEQ ID NO:3 were then made:

| | |
|---|---|
| L-(Aib)-A-E-A-K | (SEQ ID NO: 4) |
| L-I-(Nme-A)-E-A-K | (SEQ ID NO: 5) |
| L-(Abu)-A-E-A-K | (SEQ ID NO: 6) |
| G-G-Q-I-A-N-I | (SEQ ID NO: 7) |
| E-G-I-A-G-K | (SEQ ID NO: 8) |
| L-I-A-D-A-K | (SEQ ID NO: 9) |
| L-I-A-N-A-K | (SEQ ID NO: 10) |
| L-I-A-E-A-A | (SEQ ID NO: 11) |
| L-I-A-Q-A-K | (SEQ ID NO: 12) |
| L-I-A-G-G-E | (SEQ ID NO: 13) |
| L-I-A-G-E-G | (SEQ ID NO: 14) |
| A-N-V-A-E-K | (SEQ ID NO: 15) |
| L-I-A-K-G-K | (SEQ ID NO: 16) |

Of the non-standard amino acids, Aib is α-amino isobutyric acid, Nme-Ala is N-methyl alanine and Abu is α-amino butyric acid.

SEQ ID NOs:4-6, which are minor variants of SEQ ID NO:2, mimicked the biological activities of TGF-β and cytomodulin as shown by the inhibition of the proliferation of Mv-1-Lu epithelial cells and increased expression of collagen I and TGF-β in HOS cells. Sample thymidine incorporation data for SEQ ID NOs:4-6 are shown in Table 1.

TABLE 1
Inhibition of Incorporation of ³H-thymidine inMV-1 Lu cells
in the Presence of Test Peptides

| Control | ³H-Radioactivity, 10.sup.3 dpm (% |
|---|---|
| (No peptides added) | Inhibition) 3.57 (−) |
| (IV) LAibAEAK (SEQ ID NO: 4) | |
| 1 mM | 2.60 (27%) |
| 5 nM | 2.31 (35%) |
| 50 nM | 1.94 (46%) |
| 100 nM | 1.44 (60%) |
| 500 nM | 1.61 (55%) |
| (V) LINmeAEAK (SEQ ID NO: 5) | |
| 1 nM | 1.61 (47%) |
| 5 nM | 1.76 (42%) |
| 50 nM | 1.60 (41%) |
| 100 nM | 1.81 (40%) |
| 500 nM | 1.42 (53%) |
| (VI) LAbuAEAK (SEQ ID NO: 6) | |
| 1 nM | 2.02 (33%) |
| 5 nM | 1.97 (35%) |

However, SEQ ID NOs:7-8 did not display significant TGF-β activity and thus, as that term is defined herein, and by the assays used, would not be defined as TGF-β mimics since, although they possessed an atomic structure substantially the same as that shown in FIGS. 1A and 1B, they did not show TGF-β activity. This was not unexpected given the working model; e.g., although a glutamic acid is present in SEQ ID NO:8, it is on the N-terminal side of the β-bend and not on the C-terminal side as with cytomodulin, L1, and L2.

Inhibition of 3H-thymidine incorporation results for SEQ ID NOs:9-16 are shown in Table 2. The numbers shown at the various concentration are the ratio of the inhibition rate of the peptide being tested over the inhibition rate of cytomodulin (SEQ ID NO: 1) at the same concentration. Because cytomodulin inhibits the proliferation of MV-1-Lu cells at least as much as TGF-β, cytomodulin and not TGF-β was used as a control.

TABLE 2
(Inhib by new peptide/Inhib by cytomodulin)

| | | (Inhib by new peptide/ | | | |
|---|---|---|---|---|---|
| | | Inhib by cytomodulin) | | | |
| Peptide | | Concentration (nM) | | | |
| Composition | SEQ ID NO | 1 | 10 | 100 | 1000 |
| LIADAK | SEQ ID NO: 9 | 0.45 | 0.87 | 1.14 | 1.25 |
| LIANAK | SEQ ID NO: 10 | 1.70 | 2.00 | 1.43 | 3.05 |
| LIAEAA | SEQ ID NO: 11 | 1.16 | 1.00 | 1.17 | 1.85 |
| LIAQAK | SEQ ID NO: 12 | 0.90 | 1.10 | 0.80 | 1.56 |
| LIAGGE | SEQ ID NO: 13 | 1.10 | 1.33 | 1.45 | 1.90 |
| LIAGEG | SEQ ID NO: 14 | 0.66 | 0.85 | 1.41 | 1.88 |
| ANVAEK | SEQ ID NO: 15 | — | 0.80 | 1.00 | — |
| LIAKGK | SEQ ID NO: 16 | — | 0.65 | 0.67 | — |

As illustrated by Table 2, all peptides with sequences represented by SEQ ID NOs:9-16 inhibited at least some amount of thymidine uptake. Thus, all peptides with sequences represented by SEQ ID NOs:9-16 are considered TGF-β mimics.

Further analogs were constructed (SEQ ID NOS: 17-42):

| | |
|---|---|
| SEQ ID NO 17: | Gly Thr Pro Gly Pro Gln Gly Ile Ala |
| | Gly Gln Arg Gly Val Val |
| SEQ ID NO 18: | Ile Xaa Ala Glu Ala Lys |
| SEQ ID NO 19: | Leu Xaa Ala Glu Ala Lys |
| SEQ ID NO 20: | Leu Pro Ala Glu Ala Lys |
| SEQ ID NO 21: | Leu Ile Pro Glu Ala Lys |
| SEQ ID NO 22: | Leu Ile Xaa Glu Ala Lys |
| SEQ ID NO 23: | Leu Ile Ala Xaa Glu Ala |
| SEQ ID NO 24: | Ile Trp Gly Leu Asp Gly Xaa Lys |
| SEQ ID NO 25: | Trp Ile Ala Leu Glu Gly Xaa Lys |
| SEQ ID NO 26: | Gly Pro Gln Gly Ile Ala Gly Gln Arg |
| SEQ ID NO 27: | Gln Gly Ile Ala Gly Gln |
| SEQ ID NO 28: | Gln Gly Ile Ala Gly Gln Arg |
| SEQ ID NO 29: | Phe Gly Ile Ala Gly Phe |
| SEQ ID NO 30: | Gly Ile Ala Gly Gln |
| SEQ ID NO 31: | GIn Gly Ala Ile Ala Gln |
| SEQ ID NO 32: | Phe Gly Ile Ala Gly Phe |
| SEQ ID NO 33: | Gys Gly Ile Ala Gly Cys |
| SEQ ID NO 34: | Glu Gly Ile Ala Gly Lys |
| SEQ ID NO 35: | Xaa Ile Ala Ala |
| SEQ ID NO 36: | Ile Ala Xaa |
| SEQ ID NO 37: | Xaa Ile Ala Xaa |
| SEQ ID NO 38: | Ile Ile Xaa Glu Ala Lys |
| SEQ ID NO 39: | Leu Ile Xaa Glu Ala Lys |
| SEQ ID NO 40: | Leu Ile Ala Xaa Ala Lys |
| SEQ ID NO 41: | Leu Ile Ala Pro Xaa Ala |
| SEQ ID NO 42: | Leu Ile Ala Xaa Ala Lys |

Tests for bioactivity were conducted as described above for DNA inhibition. In summary, the cytomodulin analogs listed in Table 3 were all found active as agonists and thus, as defined herein, are considered TGF-β mimics.

TABLE 3

| | | |
|---|---|---|
| SEQUENCE | SEQUENCE SYMBOL | SEQ ID NO |

| | | |
|---|---|---|
| Ala-Asn-Val-Ala-Glu-Asn-Ala | A-N-V-A-E-N-A | 1 |
| Leu-Ile-Ala-Pro-Glu-Ala | L-I-A-P-E-A | 3 |
| Leu-Ile-Ala-Glu-Ala-Lys | L-I-A-E-A-K | 2 |
| IIe-Aib-Ala-Glu-Ala-Lys | I-(Aib)-A-E-A-K | 18 |
| Ile-(Ile)-(Nme-Ala)-Glu-Ala-Lys | I-(I)-(NMeA)-E-A-K | 38 |
| Leu-(Abu)-Ala-Glu-Ala-Lys | L-(Abu)-A-E-A-K | 19 |
| Leu-Ile-Ala-Asn-Ala-Lys | L-I-A-N-A-K | 10 |
| Leu-Ile-Ala-Glu-Ala-Ala | L-I-A-E-A-A | 11 |
| Leu-Ile-Ala-Lys-Gly-Lys | L-I-A-K-G-K | 16 |
| Leu-Pro-Ala-Glu-Ala-Lys | L-P-A-E-A-K | 20 |
| Leu-Ile-Pro-Glu-Ala-Lys | L-I-P-E-A-K | 21 |
| Leu-Ile-(Aib)-Glu-Ala-Lys | L-I-(Aib)-E-A-K | 22 |
| Leu-Ile-(D-Ala)-Glu-Ala-Lys | L-I-(D-Ala)-E-A-K | 39 |
| Leu-Ile-Ala-(D-Glu)-Ala-Lys | L-I-A-(D-Glu)-A-K | 40 |
| Leu-Ile-Ala-(Aib)-Glu-Ala | L-I-A-(Aib)-E-A | 23 |
| Leu-Ile-Ala-Pro-(D-Glu)-Ala | L-I-A-P-(D-Glu)-A | 41 |
| Leu-Ile-Ala-(X.sub.1)-Ala-Lys | L-I-A-(X.sub.1)-A-K | 42 |
| Ile-Trp-Gly-Leu-Asp-Gly-bAla-Lys | I-W-G-L-D-G-(bAla)-K | 24 |
| Trp-Ile-Ala-Leu-Glu-Gly-bAla-Lys | W-I-A-L-E-G-(bAla)-K | 25 |

(Abu) = α-amino butyric acid
(Aib) = α-amino isobutyric acid
(NmeA) = N-methyl alanine
X1 = trans-4-hydroxyproline

Similar testing to that described herein, and otherwise available to those of skill in the art to determine TGF-β-like activity, may be applied by those of skill in the art to peptides that are not included in the above analyses to determine whether or not they are TGF-β mimics.

### Example 4

Treatment of Dermatitis

A 68-year old male had suffered from contact dermatitis on both hands for approximately 5 to 6 years. Signs of dermatitis, such as rash, itching, crustiness, thin skin (steroids) when flexed hand would start bleeding where lines, were apparent over approximately 100 cm² of the right and left hands, respectively (all of palm and fingers). The individual applied a lotion comprising cytomodulin (SEQ ID NO: 1) at a concentration of about approximately 0.01% once per day at night. The amount applied was about 0.25 ml per hand. After 3-4 days healing of the dermatitis was apparent. In approximately two weeks the dermatitis was completely healed. No negative effects of application of the lotion were observed

### Example 5

Human Incisional Wound Healing

A 69 year old male sustained a deep incisional wound on the hand as the result of an accident while opening a can. The wound was about 4 cm in length, and an average of approximate 4 mm in depth. The wound penetrated both the epidermis and the dermis as well as underlying tissue, as in some places underlying ligaments were visible. An emergency room surgeon sewed up wound within 1 h after injury. The wound was closed with one set of subcutaneous stitches and one set of stitches in the skin. The surgeon stated that the stitches should remain in for 10-12 days. The wound bled extensively even after stitching.

On Day 2, and for the next five days, a CM-1 suspension in generic hand cream (40 micrograms/ml, SEQ ID NO: 1), was applied to the stitched wound daily with dressing change. Approximately 0.5 ml was used in each application (about 20 micrograms of CM-1). A thin layer of the suspension was applied to the wound and wound edges. The hand was heavily bandaged and kept in sling for four days. Photographs were taken periodically at the time of dressing change. Within 6 days of the first application of CM-1 suspension (7 days post-injury) the wound had closed and the stitches were removed; this was approximately four to six days earlier than the surgeon had recommended. The wound had closed completely at this point. At this time the location of the incision was still visible. However, the wound continued to heal and by Day 10, the location of the incision was no longer visible to the naked eye (i.e., the wound had healed with no visible scarring, despite the fact that it had penetrated the dermis). The wound healed completely and the hand returned to total function.

This Example demonstrates that application of CM-1 to an incisional wound enhances wound healing and results in no visible scar formation, even though the dermis is completely penetrated by the wound.

### Example 6

Treatment of Insect Bites and Minor Cuts and Burns.

A 59 yr old, Caucasian female reported that she was very allergic to insect bites, which usually made a large red welt, then turned purple and looked like a severe bruise, lasting for about a week. The subject applied a lotion comprising cytomodulin (SEQ ID NO: 1) at a concentration of about 0.001% to an insect bite and noted that the bite disappeared by the next day. The subject also tried it on minor cuts and burns with the result that healing took place in a day or two. The subject reported that there were no adverse effects.

### Example 7

Treatment of Acne.

A 48 yr old Caucasian female applied a lotion comprising cytomodulin (SEQ ID NO: 1) at a concentration of about 0.001% to acne on lower right side of face near mouth, daily. Within 24 hours the acne started to clear. Within one week the acne was gone with no significant scarring. No adverse effects were noted.

### Example 8

Treatment of Contact Dermatitis.

A 15 yr old Caucasian female developed a sensitivity to the lipstick she was using and her lips as well as the edge around the lips (½ cm. approx.) became very red and chapped. She applied a lotion comprising cytomodulin (SEQ ID NO: 1) at a concentration of about 0.001% to the irritated area. Within 24 hours the irritation had subsided nearly completely. No adverse effects were noted.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-31. (canceled)

32. A method of treatment comprising administering to an individual in need thereof an effective amount of a composition comprising a peptide in a vehicle at a concentration of about 0.0001% to about 0.01% by weight, wherein the peptide comprises a sequence selected from the group consisting of SEQ ID NOS: 2, 3, 4, 5, 6, 10, 11, 13, 14, and 15, wherein the individual suffers from a condition selected from the group consisting of dermatitis, psoriasis, acne, urticaria, mucositis, and a wound.

33. The method of claim 32 wherein the peptide is administered topically.

34. The method of claim 33 wherein the peptide is administered in a vehicle comprising a lotion.

35. The method of claim 34 wherein the lotion comprises water, mineral oil, a polyethylene glycol, and lanolin alcohol.

36. The composition of claim 32 wherein the peptide is present at a concentration of about 0.001% to about 0.01%.

37. The composition of claim 32 wherein the peptide is present at a concentration of about 0.001%.

38. The composition of claim 32 wherein the peptide is present at a concentration of about 0.01%.

39. The composition of claim 32 wherein the vehicle comprises water, mineral oil, a polyethylene glycol, and lanolin alcohol.

40. The method of treatment of claim 32 wherein the individual suffers from dermatitis.

41. The method of treatment of claim 32 wherein the individual suffers from psoriasis.

42. The method of treatment of claim 32 wherein the individual suffers from acne.

43. The method of treatment of claim 32 wherein the individual suffers from urticaria.

44. The method of treatment of claim 32 wherein the individual suffers from mucositis.

45. The method of treatment of claim 32 wherein the individual suffers from a wound.


SEQUENCE LISTING (44 sequences)

SEQ ID NO: 1 - protein, 7 aa, Artificial Sequence
  Description of Artificial Sequence Synthetic peptide
ANVAENA

SEQ ID NO: 2 - protein, 6 aa, Artificial Sequence
  Description of Artificial Sequence Synthetic peptide
LIAEAK

SEQ ID NO: 3 - protein, 6 aa, Artificial Sequence
  Description of Artificial Sequence Synthetic peptide
LIAPEA

SEQ ID NO: 4 - protein, 6 aa, Artificial Sequence
  Description of Artificial Sequence Synthetic peptide
  MOD_RES (2): alpha-amino isobutyric acid
LXAEAK

SEQ ID NO: 5 - protein, 6 aa, Artificial Sequence
  Description of Artificial Sequence Synthetic peptide
  MOD_RES (3): N-methyl alanine
LIXEAK

SEQ ID NO: 6 - protein, 6 aa, Artificial Sequence
  Description of Artificial Sequence Synthetic peptide
  MOD_RES (2): alpha-amino butyric acid
LXAEAK

SEQ ID NO: 7 - protein, 7 aa, Artificial Sequence
  Description of Artificial Sequence Synthetic peptide
GGQIANI

SEQ ID NO: 8 - protein, 6 aa, Artificial Sequence
  Description of Artificial Sequence Synthetic peptide
EGIAGK

SEQ ID NO: 9 - protein, 6 aa, Artificial Sequence
  Description of Artificial Sequence Synthetic peptide
LIADAK

SEQ ID NO: 10 - protein, 6 aa, Artificial Sequence
  Description of Artificial Sequence Synthetic peptide
LIANAK

SEQ ID NO: 11 - protein, 6 aa, Artificial Sequence
  Description of Artificial Sequence Synthetic peptide
LIAEAA

SEQ ID NO: 12 - protein, 6 aa, Artificial Sequence
  Description of Artificial Sequence Synthetic peptide
LIAQAK

SEQ ID NO: 13 - protein, 6 aa, Artificial Sequence
  Description of Artificial Sequence Synthetic peptide
LIAGGE

SEQ ID NO: 14 - protein, 6 aa, Artificial Sequence
  Description of Artificial Sequence Synthetic peptide
LIAGEG

SEQ ID NO: 15 - protein, 6 aa, Artificial Sequence
  Description of Artificial Sequence Synthetic peptide
ANVAEK

SEQ ID NO: 16 - protein, 6 aa, Artificial Sequence
  Description of Artificial Sequence Synthetic peptide
LIAKGK

SEQ ID NO: 17 - protein, 15 aa, Artificial Sequence
  Description of Artificial Sequence Synthetic peptide
GTPGPQGIAGQRGVV

SEQ ID NO: 18 - protein, 6 aa, Artificial Sequence
  Description of Artificial Sequence Synthetic peptide
  MOD_RES (2): alpha-amino isobutyric acid
IXAEAK

SEQ ID NO: 19 - protein, 6 aa, Artificial Sequence
  Description of Artificial Sequence Synthetic peptide
  MOD_RES (2): alpha-amino butyric acid
LXAEAK

SEQ ID NO: 20 - protein, 6 aa, Artificial Sequence
  Description of Artificial Sequence Synthetic peptide
LPAEAK

SEQ ID NO: 21 - protein, 6 aa, Artificial Sequence
  Description of Artificial Sequence Synthetic peptide
LIPEAK

SEQ ID NO: 22 - protein, 6 aa, Artificial Sequence
  Description of Artificial Sequence Synthetic peptide
  MOD_RES (3): alpha-amino isobutyric acid
LIXEAK

SEQ ID NO: 23 - protein, 6 aa, Artificial Sequence
  Description of Artificial Sequence Synthetic peptide
  MOD_RES (4): alpha-amino isobutyric acid
LIAXEA

SEQ ID NO: 24 - protein, 8 aa, Artificial Sequence
  Description of Artificial Sequence Synthetic peptide
  MOD_RES (7): b-Alanine
IWGLDGXK

SEQ ID NO: 25 - protein, 8 aa, Artificial Sequence
  Description of Artificial Sequence Synthetic peptide
  MOD_RES (7): b-Alanine
WIALEGXK

SEQ ID NO: 26 - protein, 9 aa, Artificial Sequence
  Description of Artificial Sequence Synthetic peptide
GPQGIAGQR

SEQ ID NO: 27 - protein, 6 aa, Artificial Sequence
  Description of Artificial Sequence Synthetic peptide
QGIAGQ

SEQ ID NO: 28 - protein, 7 aa, Artificial Sequence
  Description of Artificial Sequence Synthetic peptide
QGIAGQR

SEQ ID NO: 29 - protein, 6 aa, Artificial Sequence
  Description of Artificial Sequence Synthetic peptide
FGIAGF

SEQ ID NO: 30 - protein, 5 aa, Artificial Sequence
  Description of Artificial Sequence Synthetic peptide
GIAGQ

SEQ ID NO: 31 - protein, 6 aa, Artificial Sequence
  Description of Artificial Sequence Synthetic peptide
QGAIAQ

SEQ ID NO: 32 - protein, 6 aa, Artificial Sequence
  Description of Artificial Sequence Synthetic peptide
FGIAGF

SEQ ID NO: 33 - protein, 6 aa, Artificial Sequence
  Description of Artificial Sequence Synthetic peptide
CGIAGC

SEQ ID NO: 34 - protein, 6 aa, Artificial Sequence
  Description of Artificial Sequence Synthetic peptide
EGIAGK

SEQ ID NO: 35 - protein, 4 aa, Artificial Sequence
  Description of Artificial Sequence Synthetic peptide
  MOD_RES (1): variable amino acid
XIAA

SEQ ID NO: 36 - protein, 3 aa, Artificial Sequence
  Description of Artificial Sequence Synthetic peptide
  MOD_RES (3): variable amino acid
IAX

SEQ ID NO: 37 - protein, 4 aa, Artificial Sequence
  Description of Artificial Sequence Synthetic peptide
  MOD_RES (1): variable amino acid
  MOD_RES (4): variable amino acid
XIAX

SEQ ID NO: 38 - protein, 6 aa, Artificial Sequence
  Description of Artificial Sequence Synthetic peptide
  MOD_RES (3): N-methyl alanine
IIXEAK

SEQ ID NO: 39 - protein, 6 aa, Artificial Sequence
  Description of Artificial Sequence Synthetic peptide
  MOD_RES (3): D-alanine
LIXEAK

SEQ ID NO: 40 - protein, 6 aa, Artificial Sequence
  Description of Artificial Sequence Synthetic peptide
  MOD_RES (4): D-glutamic acid
LIAXAK

SEQ ID NO: 41 - protein, 6 aa, Artificial Sequence
  Description of Artificial Sequence Synthetic peptide
  MOD_RES (5): D-glutamic acid
  MOD_RES (5): variable amino acid
LIAPXA

SEQ ID NO: 42 - protein, 6 aa, Artificial Sequence
  Description of Artificial Sequence Synthetic peptide
  MOD_RES (4): trans-4-hydroxyproline
  MOD_RES (4): variable amino acid
LIAXAK

SEQ ID NO: 43 - protein, 4 aa, Artificial Sequence
  Description of Artificial Sequence Synthetic peptide
LIAE

SEQ ID NO: 44 - protein, 4 aa, Artificial Sequence
  Description of Artificial Sequence Synthetic peptide
NVAE